(19) United States
(12) Patent Application Publication
(10) Pub. No.: US 2023/0257653 A1
(43) Pub. Date: Aug. 17, 2023

(54) FRACTAL FORMULATIONS

(71) Applicant: NANOGYRE Sprl., Liège (BE)

(72) Inventor: Jallal Messadek, Liège (BE)

(73) Assignee: NANOGYRE Sprl, Liège (BE)

(21) Appl. No.: 18/304,497

(22) Filed: Apr. 21, 2023

Related U.S. Application Data
(63) Continuation-in-part of application No. PCT/EP2020/025468, filed on Oct. 21, 2020.

(51) Int. Cl. C09K 19/06

(52) U.S. Cl. CPC C09K 19/061; B82Y 30/00

(57) ABSTRACT

Glycine betaine based liquid crystal comprising in dry matter at least:
80 to 99.5% by weight of glycine betaine, and
0.5 to 20% by weight of a non-ionic water soluble tensioactive system having a HLB value equal to 12 or more than 12,
   whereby the weight ratio glycine betaine/non-ionic water soluble tensioactive system is greater than 10,
   said glycine betaine and said non-ionic water soluble tensioactive being solubilized in water for forming the glycine betaine based liquid crystal.

The present application is a Continuation-in-Part of International application PCT/EP2020/025468 filed on Oct. 21, 2020 and published under number WO 2021/078412 on Apr. 29, 2021. The International application was filed in the name of the inventor MESSADEK Jallal and was assigned to NANOGYRE Sprl.

### FIELD OF THE INVENTION

The invention relates to a glycine betaine based liquid crystal comprising in dry matter essentially glycine betaine, enabling to increase the water solubility of hydrophobic entities, especially having a very low water solubility.

### STATE OF THE ART

Currently, the majority of the new pharmaceutical molecules discovered are lipophilic and have a low solubility in water, which leads to a low bioavailability, the production of intra- and interindividual variations and an absence of a proportional effect of the concentrations of pharmaceutical active.

The low water-soluble power of these active molecules is a clearance for the development of solid and effective oral compositions, in particular in terms of formulation, bioavailability, control of the release of the active and of marketing of new pharmaceutical products.

The LFCS (Lipid Formulation Classification System) classification system Pouton's works (Eur J Pharm Sci 2006; 29: 275-87) have made it possible to place a system for classifying these lipid formulations (LFCS or Lipid formulation classification system), with the aim of harmonizing their understanding. Thus, according to the LFCS, the lipid formulations can be classified into four different categories: types I, II, III (A and B) and IV (Table 1):
Type I: consisting of mono-, di- or triglycerides in which the active substance is dissolved. These systems must be digested by the open gastric enzymes to form amphiphilic products and thus keep the active substance dissolved in the gastrointestinal fluids.
Type II: these formulations integrate 20 to 60% of surfactants with a hydrophilic/lipophilic balance (Hydrophilic Lipophilic Balance, HLB) of less than 12. They constitute in particular self-emulsifying systems (Self-Emulsifying Drug Delivery Systems, SEDDS), forming oil-in-water emulsions with low stirring and independently of enzymatic digestion. Typically, SEDDS form opaque emulsions with emulsion droplets of size greater than 100 nm.
Type III: containing a lipid part, hydrophilic surfactants (HLB>12) and co-solvents, they can form SEDDS but also SMEDDS (Self-Micro Emulsifying Drug Delivery Systems) or SNEDDS (Self-Nano Emulsifying Drug Delivery Systems). The emulsions formed by the SMEDDS have particle sizes of less than 100 nm and are opalescent or transparent. The nanoemulsions formed by the SNEDDS are transparent with droplets less than 10 nm. Among type III formulations, types III a, having a lipid part of 40% to 80%, types III b, which are more soluble in water (less than 20% of oil) are distinguished. The latter have a better dispersion but an increased risk of precipitation of the API, due to the large proportion of hydrophilic co-solvent (20 to 50%).
Type IV: free of lipid part, these systems consist only of surfactants that are predominantly hydrophilic, and of co-solvents. They make it possible to dissolve a large amount of active substance and thus to increase the dose charged in the formulation. The dispersion in water of formulations of type IV forms micellar solutions of clear appearance.

TABLE 1

| | Formulations Constitutions (weight %) | | | | |
|---|---|---|---|---|---|
| Excipients | Type I | Type II | Type III a | Type III b | Type IV |
| Oils: mono, di or | 100 | 40-80 | 80-40 | <20 | — |
| triglycerides | | | | | |
| Water Insoluble Surfactant | — | 20-60 | — | — | 0-20 |
| (HLB < 12) | | | | | |
| Water Soluble Surfactant | — | — | 20-40 | 20-50 | 30-80 |
| (HLB > 12) | | | | | |
| Hydrophilic Cosolvents (ex: | — | — | 0-40 | 20-50 | 0-50 |
| PEG, propylene-glycol, | | | | | |
| Transcutol ®) | | | | | |

### Liquid Crystals

Basic physics teaches that the states of matter are gaseous, liquid and solid states.

However, certain organic substances do not have a single transition between the liquid and solid state but several transitions causing intermediate states to appear: the mesophases or liquid crystals.

A solid (crystal) is characterized by a regular stack of molecules, atoms or ions on a periodic network in the three spatial directions. The position correlations are said to be long-range.

On the contrary, a liquid has only a short-range order which is exponentially stamped with the distance.

Liquid crystals have an order in orientation or long-range position while retaining a disorder of the liquid type in at least one of the directions of the space. They are therefore systems having a number of intermediate symmetries between those of the solid and liquid phases.

There are many mesophases that differ by the type and degree of self-organization of the molecules. This collective directional behavior depends on the nature and structure of the mesogens but also a certain number of external parameters such as mechanical, electrical or magnetic forces.

Consequently, a liquid crystal is a state of the material which combines properties of a conventional liquid and those of a crystallized solid. Its state is expressed by the term of mesophase or mesomorphic state (from Greek “intermediate form”). The nature of the mesophase differs depending on the nature and structure of the mesogen, molecule at the origin of the mesophase, as well as temperature, pressure and concentration conditions.

There are two large classes of liquid crystals: thermotropic liquid crystals and lyotropic liquid crystals. Thermotropes change phase as a function of temperature while lyotropes are substances in which the mesophases are induced by the presence of a solvent and depend on the concentration of the solvent, as on temperature.

So-called amphotropes, the molecules making it possible to generate thermotropic phases as well as lyotropic phases in the presence of solvent.

The different mesophases can also be distinguished by their different optical properties, such as birefringence. In general, the liquid crystals by interacting with the light show remarkable and different optical properties from those of a simple liquid phase.

The properties of the liquid crystals are dependent on the temperature and the presence or absence of solvent, and as a function of these properties, form two classes: thermotropic liquid crystals (TLC) and lyotropic liquid crystals (LLC).

In thermotropic liquid crystals, the shape of the molecules dictates the order of orientation and the thermal movement gives the mobility.

The lyotropic liquid crystals are governed by the relative concentration of the solute. A lyotropic liquid crystal is composed of two or more components which exhibit liquid crystalline properties in certain concentration ranges.

Unlike thermotropic liquid crystals, lyotropes have an additional degree of freedom of concentration which allows them to induce a variety of different phases.

However, this distinction between thermotropic and lyotropes would not be complete without mention of the amphitropic liquid crystals which have both thermotropic and lyotropic properties.

Many organic substances do not have a single transition between the liquid and solid state but several transitions revealing intermediate states: the mesophases or more commonly liquid crystals.

The liquid crystal classification scheme divides them into three different types of mesogens, based on the level of order of the molecules in the material mass. This structural classification is based on criteria of order and symmetry in molecular arrangements and comprises three liquid crystal phases:
The nematic phase
The cholesteric phase
The smectic phase

In addition to the preceding classification, a second characterizes the different phases of the different states and organizations of the liquid crystals:
Discontinuous cubic phase (micellar phase).
Hexagonal cylindrical phase (intermediate phase).
Cubic phase bi-continuous.
Lamellar phase (net phase)
Inversion of the bi-continuous cubic phase.
Inverting the hexagonal cylindrical phase.
Reverse cubic phase (reverse micellar phase)

They are therefore systems having a number of intermediate symmetries between those of the solid and liquid phases. There are many mesophases that differ by the type and degree of self-organization of the molecules.

This collective directional behavior, called a director, depends on the nature and the structure of the mesogens but also a certain number of external parameters such as the mechanical and/or thermodynamic and/or chemical and/or electrical and/or optical and/or magnetic forces.

The different mesophases of the liquid crystals can be distinguished by optical properties such as birefringence. In a polarized light microscope, a liquid crystal material will appear to be composed of zones of distinct texture. Each zone corresponds to a domain where the molecules are oriented in a different direction.

### The BCS System

The Biopharmaceuticals Classification System or BCS, is a system making it possible to differentiate the active principles as a function of their solubility and their permeability.

This system restricts the prediction using the solubility and intestinal permeability parameters. The classification of the solubility is based on the American Pharmacopoeia, whereas that of the intestinal permeability is based on a comparison with the intravenous injection. All these factors are very important because 85% of the most sold drugs in the United States and in Europe are administered orally.

According to the Biopharmaceuticals Classification System (BCS), the medicinal substances are classified into four classes according to their solubility and their permeability:
Class I—High Permeability, High Solubility—These compounds are well absorbed and their absorption rate is generally greater than that of the excretion.
Class II—High permeability, low solubility—The bioavailability of these products is limited by their solvating rate. A correlation between the in vivo bioavailability and the in vitro solvation can be found.
Class III—Low permeability—The absorption is limited by the permeation rate but the drug is solvated very rapidly. If the formulation does not modify the permeability or the gastrointestinal duration, Class I criteria can be applied.
Class IV—Low permeability, low solubility—These compounds have poor bioavailability. Usually, they are not well absorbed by the intestinal mucosa and a strong variability is expected, this is due to the fact that a small variation in bioavailability results in a large relative variation thereof.

In the BCS system, the active principles are classified on the basis of the solubility, the dissolution (speed of solubilization) and the permeability.

The definition of the solubility class is based on the highest dose of an immediate release product. A drug is considered to be highly soluble when the highest dose is soluble in 250 ml or less of aqueous medium in a pH range of 1 to 7.5. The 250 ml volume estimate is derived from typical bioequivalence study protocols which prescribe the administration of a drug product to young human volunteers with a water glass.

To define the dissolution, an immediate release product is considered to dissolve rapidly when at least 85% of the labeled amount of the drug substance dissolves in 15 minutes using the USP dissolution apparatus 1 at 100 rpm or the apparatus 2 at 50 rpm in a volume of 900 ml or less in the following media:
M HCl or simulated gastric fluid without enzymes
Buffer pH 4.5
Buffer pH 6.8 or simulated intestinal liquid without enzymes.

The HLB is a method, proposed in 1949 by Griffin, which makes it possible to number the equilibrium existing between the hydrophilic part and the lipophilic part of the surfactant molecule, equilibrium linked to the solubility in water. The scale varies from 0 to 20: the higher the value, the greater the solubility in water.

Log P, also called Log Kow, is a measurement of the differential solubility of chemical compounds in two solvents (octanol/water partition coefficient).

Log P is equal to the logarithm of the ratio of the concentrations of the substance studied in octanol and in water. Log P=Log (Coct/Cwater), in which Coct is the concentration of the substance in octanol, while Cwat is the concentration of the substance in water. This value makes it possible to grasp the hydrophilic or hydrophobic (lipophilic) characteristic of a molecule.

Indeed, if Log P is positive and very high, this expresses the fact that the molecule under consideration is much more soluble in octanol than in water, which reflects its lipophilic character, and vice versa, if Log P is negative, this means that the molecule under consideration is hydrophilic. A zero Log P means that the molecule is also soluble in a solvent than in the other.

Said document teaches a skin cleaning composition in a liquid crystal state. Said composition comprises: (a) 15 to 45% by weight higher fatty acid salt, (b) 7 to 25% by weight of a betaine type amphoteric surfactant, (c) 1 to 5% by weight polyoxyethylene nonionic surfactant, and (d) 15 to 35% by weight of polyhydric alcohol.

As stated in said document in case the composition comprises less than 15% by weight of higher fatty acid salt, there is no liquid crystal, while the composition comprises more than 25% by weight of betaine type amphoteric surfactant, a precipitation occurs.

Said document teaches thus away the present invention relating to a glycine betaine based liquid crystal comprising essentially glycine betaine CAS number 107-43-7 and the tensioactive system with a HLB equal to or greater than 12, the weight ratio glycine betaine/tensioactive system being greater than 10:1.

### BRIEF DESCRIPTION OF THE INVENTION

The invention relates to a glycine betaine based liquid crystal comprising in dry matter at least:
80 to 99.5% by weight (advantageously 90 to 99.5% by weight, preferably 95 to 99.5% by weight) of glycine betaine of formula (CH₃)₃N⁺CH₂COO⁻, CAS number: 107-43-7, and
0.5 to 20% by weight of a non-ionic water soluble tensioactive system selected from the group consisting of non-ionic water soluble tensioactive agents having a HLB value equal to 12 or more than 12 and mixtures of non-ionic water soluble tensioactive agents having each a HLB value equal to 12 or more than 12,
   whereby the weight ratio glycine betaine/non-ionic water soluble tensioactive system is greater than 10:1, said glycine betaine and said non-ionic water soluble non-ionic water soluble tensioactive being solubilized in water with a weight ratio glycine betaine/water comprised between 1:3 to 3:1 for forming the glycine betaine based liquid crystal.

According to advantageous embodiments, the glycine betaine based liquid crystal has one or more of the following characteristics:
The glycine betaine based liquid crystal has a particle size of less than 350 nanometers, such as less than 300 nanometers, advantageously less than 250 nanometers, such as less than 200 nanometers, or even such as less than 150 nanometers, preferably less than 100 nanometers, such as less than 80 nanometers, or even less than 60 nanometers, for example less than 50 nanometers, most preferably less than 40 nanometers, such as less than 20 nanometers or even less than 10 nanometers. The glycine betaine based liquid crystal can comprise a mix of different particle size.
The weight ratio glycine betaine/non-ionic water soluble tensioactive system is comprised between 20:1 and 50:1.
The non-ionic water soluble tensioactive system is selected from the group consisting of non-ionic water soluble tensioactive agents having a HLB value equal to or more than 13 and mixtures of non-ionic water soluble tensioactive agents having each a HLB value equal to or more than 13.
The non-ionic water soluble tensioactive system is selected from the group consisting of Tween 20 (E432-HLB 15), Tween 80 (E433-HLB 15), Tween 40 (E434-HLB 15.6), Tween 60 (E435-HLB 14.9), Tween 65 (E436-HLB 10.5), Quillaja Saponaria Molina (E999), as well as their mixtures.
The glycine betaine based liquid crystal being subjected to a drying step for reducing the water content to less than 10% by weight, advantageously less than 5% weight, such as less than 2% by weight or even less than 1% by weight. The liquid crystal is formed in presence of water, and can then be dried so as to reduce its water content, or even to achieve a dry form.
The glycine betaine based liquid crystal further comprises a hydrophobic entity, whereby the weight ratio glycine betaine/hydrophobic entity is equal to or greater than 10:1, advantageously greater than 15:1, preferably greater than 20:1.
The hydrophobic entity has a octanol/water partition coefficient defined by a Log Kow greater than 1.5, advantageously greater than 3, such as greater than 4, preferably greater than 5, or even greater than 6.
The hydrophobic entity is selected from the group consisting of dietetic substances, cosmetic substances, nutrients and pharmaceutical substances.
The glycine betaine based liquid crystal is substantially free of organic solvent, such as ethanol, methanol, DSMO, propanol, butanol, octanol, chloroform.
a combination of two or more of such characteristics.

The invention relates also to a composition for administering to a mammal a hydrophobic entity, said composition comprising:
glycine betaine based liquid crystals comprising in dry matter at least:
80 to 99.5% by weight of glycine betaine of formula (CH₃)₃N⁺CH₂COO⁻, CAS number: 107-43-7,
0.5 to 20% by weight of a non-ionic water soluble tensioactive system selected from the group consisting of non-ionic water soluble tensioactive agents having a HLB value equal to 12 or more than 12 and mixtures of non-ionic water soluble tensioactive agents having each a HLB value equal to 12 or more than 12, and
a quantity of the hydrophobic entity,
   whereby the weight ratio glycine betaine/non-ionic water soluble tensioactive system is greater than 10:1, and whereby the weight ratio glycine betaine/hydrophobic entity is equal to or greater than 5:1, said glycine betaine and said non-ionic water soluble tensioactive being solubilized in water with a weight ratio glycine betaine/water comprised between 1:3 to 3:1 for forming the glycine betaine based liquid crystal (which can thereafter be dried).

The glycine betaine based liquid crystals have advantageously one or more characteristics of the glycine betaine based liquid crystal of the invention disclosed here above.

Among others, the composition of the invention has advantageously one or more of the following characteristics:
the glycine betaine based liquid crystals have each a particle size of less than 350 nanometers, advantageously less than 100 nanometers, preferably less than 20 nanometers. The composition can comprise a mix of liquid crystals having different particle sizes.
the weight ratio glycine betaine/non-ionic water soluble tensioactive system of the glycine betaine based liquid crystal is comprised between 20:1 and 50:1.
the non-ionic water soluble tensioactive system of the glycine betaine based liquid crystal is selected from the group consisting of non-ionic water soluble tensioactive agents having a HLB value equal to or more than 14 and mixtures of non-ionic water soluble tensioactive agents having each a HLB value equal to 13 or more than 13.
the non-ionic water soluble tensioactive system of the glycine betaine based liquid crystal is selected from the group consisting of Tween 20 (E432-HLB 15), Tween 80 (E433-HLB 15), Tween 40 (E434-HLB 15.6), Tween 60 (E435-HLB 14.9), Tween 65 (E436-HLB 10.5), Quillaja Saponaria Molina (E999), as well as their mixtures.
The composition has a water content of less than 10% by weight, advantageously less than 1%. The composition is preferably dry.
the hydrophobic entity has an octanol/water partition coefficient defined by a Log Kow greater than 1.5, advantageously greater than 3.
The composition is substantially free of organic solvent.
The composition is in a form selected from the group consisting of
   (a) ready to use administration forms selected from the group consisting of oral formulations, sublingual formulations, rectal formulations, parenteral formulations, topic formulations, injectable formulations, mucosal formulations, nasal formulations, cutaneous formulations, subcutaneous formulations, transcutaneous formulations, transdermal formulations, pulmonary formulations, eye drop formulations, and ready to use administration forms for preparing formulation, and
   (b) intermediate administration forms adapted to be mixed with water for preparing at least one ready to use administration forms selected from the group consisting of
      oral formulations, sublingual formulations, rectal formulations, parenteral formulations, topic formulations, injectable formulations, mucosal formulations, nasal formulations, cutaneous formulations, subcutaneous formulations, transcutaneous formulations, transdermal formulations, pulmonary formulations, eye drop formulations.

The invention further relates to a process for the preparation of a glycine betaine based liquid crystal of the invention comprising in dry matter at least:
80 to 99.5% by weight of glycine betaine of formula (CH₃)₃N⁺CH₂COO⁻, CAS number: 107-43-7, and
0.5 to 20% by weight of a non-ionic water soluble tensioactive system selected from the group consisting of non-ionic water soluble tensioactive agents having a HLB value equal to 12 or more than 12 and mixtures of non-ionic water soluble tensioactive agents having each a HLB value equal to 12 or more than 12,
   whereby the weight ratio glycine betaine/non-ionic water soluble tensioactive system is greater than 10:1.
      said process comprising at least the following steps:
a preparation step of an aqueous medium comprising water, the glycine betaine and the non-ionic water soluble tensioactive system, whereby in dry matter the aqueous medium comprises at least:
   80 to 99.5% by weight of glycine betaine of formula (CH₃)₃N⁺CH₂COO⁻, CAS number: 107-43-7, and
   0.5 to 20% by weight of a non-ionic water soluble tensioactive system selected from the group consisting of non-ionic water soluble tensioactive agents having a HLB value equal to 12 or more than 12 and mixtures of non-ionic water soluble tensioactive agents having each a HLB value equal to 12 or more than 12,
   whereby the weight ratio glycine betaine/non-ionic water soluble non-ionic water soluble tensioactive system is greater than 10:1, said glycine betaine and said non-ionic water soluble tensioactive being solubilized in water with a weight ratio glycine betaine/water comprised between 1:3 to 3:1, and
a mixing step of the aqueous medium for forming the glycine betaine based liquid crystal.

The process of the invention has advantageously one or more of the following characteristics:
the mixing step is operated for generating glycine betaine based liquid crystals with a particle size of less than 350 nanometers,
the mixing step is operated for generating glycine betaine based liquid crystals with a particle size of less than 100 nanometers,
the weight ratio glycine betaine/non-ionic water soluble tensioactive system in the aqueous medium is comprised between 20:1 and 50:1.
the non-ionic water soluble tensioactive system of the aqueous medium is selected from the group consisting of non-ionic water soluble tensioactive agents having a HLB value equal to 13 or more than of more than 13 and mixtures of non-ionic water soluble tensioactive agents having each a HLB value equal to or more than 13.
the aqueous medium has a betaine content such that the weight ratio betaine/water is about 1:1.
the preparation step of the aqueous medium comprises a step of adding a hydrophobic entity, whereby the weight ratio betaine/therapeutic entity is greater than 10:1, advantageously greater than 20:1.
the aqueous medium consists of water, the glycine betaine, the non-ionic water soluble tensioactive system and the hydrophobic entity, whereby the therapeutic entity has a octanol/water partition coefficient defined by a Log Kow greater than 1.5, advantageously greater than 3.
the hydrophobic entity is selected from the group consisting of dietetic substances, cosmetic substances, nutrients and pharmaceutical substances.
the preparation step of the aqueous medium comprises a step of adding a hydrophobic entity, whereby the weight ratio betaine/therapeutic entity is greater than 10:1,
   in which the aqueous medium consists of water, the glycine betaine, the non-ionic water soluble tensioactive system and the therapeutic entity, whereby the therapeutic entity has a octanol/water partition coefficient defined by a Log Kow greater than 1.5,
   in which the therapeutic entity is selected from the group consisting of dietetic substances, cosmetic substances, nutrients and pharmaceutical substances,
   whereby the preparation step of the aqueous medium comprises the following steps:
   mixing the water, the non-ionic water soluble tensioactive system and the hydrophobic entity, so as to prepare an intermediate aqueous medium, and
   adding the glycine betaine in dry form to the intermediate aqueous medium.
The process further comprises at least one process step selected from the group consisting of:
a centrifugation step,
a shear mixing step,
a drying step for removing at least partly water of the aqueous medium,
a separation step for removing at least partly the hydrophobic entity not in a solubilized form present in the aqueous medium,
a grinding step, and
a shaping step with a carrier.

The invention further relates to a method of treatment of a mammal selected from the group consisting of:

(a) mammals suffering at least one trouble from a disease, and
(b) mammals at risk of suffering at least one trouble from a disease, by administering to said mammal at least a dose of a therapeutic entity selected from the group consisting of nutrients, pharmaceutical substances and mixtures thereof,
in which the therapeutic entity to be administered is in the form of a composition comprising glycine betaine based liquid crystals comprising in dry matter at least:
80 to 99.5% by weight of glycine betaine of formula (CH₃)₃N⁺CH₂COO⁻, CAS number: 107-43-7,
0.5 to 20% by weight of a non-ionic water soluble tensioactive system selected from the group consisting of non-ionic water soluble tensioactive agents having a HLB value equal to 12 or more than 12 and mixtures of non-ionic water soluble tensioactive agents having each a HLB value equal to 12 or more than 12, and
a quantity of the therapeutic entity,
   whereby the weight ratio glycine betaine/non-ionic water soluble tensioactive system is greater than 10:1, and whereby the weight ratio glycine betaine/therapeutic entity is equal to or greater than 5:1.

Advantageously, the therapeutic entity is an hydrophobic entity whereby its mix with the glycine betaine based liquid crystal confers to said hydrophobic entity a solubility in water which is at least 100 times higher than the solubility of the hydrophobic entity as such in water.

In the method of treatment of the invention, the composition for administering the therapeutic entity is advantageously in a form selected from the group consisting of oral formulations, sublingual formulations, rectal formulations, parenteral formulations, topic formulations, injectable formulations, mucosal formulations, nasal formulations, cutaneous formulations, subcutaneous formulations, transcutaneous formulations, transdermal formulations, pulmonary formulations, and eye drop formulations.

The methods of the invention enable one or more of the following characteristics: a better bioavailability of a drug, better efficacy, immediate release of a drug, less side effects and combinations thereof.

### BRIEF DESCRIPTION OF THE FIGURE

FIG. 1 shows the germination/nucleation of two compositions (Example 4) subjected to a drying at 60° C., Tube A of FIG. 1 relating to the nucleation germination of a composition consisting of water (2 g) and glycine betaine (2 g), while Tube B relates to the nucleation germination of a composition consisting of water (g), glycine betaine (2 g) and tensioactive system (Polysorbate 80 HLB=15).

### DESCRIPTION OF ELEMENTS OF THE INVENTION

The invention describes novel betaine structures in the form of lyotropic and/or anisotropic and/or amphotrope liquid crystals.

It has surprisingly been discovered that the mixtures in aqueous solutions and in well-defined proportions of a betaine base, preferably anhydrous and/or monohydrate, and of one or more surfactants and/or co-surfactants and/or surfactants, non-ionic & hydrophilic and having an HLB equal or greater than 12, preferably having an HLB equal or greater than 13, more preferably having an HLB equal or greater than 14, preferentially having an HLB equal or greater than 16, create new liquid betaine crystals.

These new liquid crystals demonstrate remarkable properties for increasing the solubility in water and/or in physiological liquids of extremely poorly water-soluble, or even insoluble, molecules and/or entities.

In addition, these new liquid crystals behave like nano-emulsions with the remarkable and unique feature of being completely free of oils or lipid compounds with the exception of the lipid entities that they convey and/or that they solubilize.

Under such circumstances, the invention claims the unique hybrid characteristic and/or feature of the present forms and/or of the present vectors.

Indeed, in addition to their characteristics of new liquid crystals, the present forms also constitute a new and fifth category in the LFCS classification which comprises only four.

The structures of the invention, by their lipid-free compositions, approach mostly of the LFCS type IV, with nevertheless major and fundamental differences that allow in the setup of a new type of LFCS that can be qualified as LFCS type V.

Indeed, it suffices to look at Table 2 in order to see the fundamental differences existing between the two types.

TABLE 2

| | Formulations Constitutions | |
|---|---|---|
| | (% by weight), without | |
| | water, without cargoes | |
| | | Type V of the |
| Excipients | Type IV | Invention |
| Oils: mono, di or triglycerides | — | — |
| Water Insoluble Surfactant | 0-20 | |
| (HLB < 12) | | |
| Water Soluble Surfactant | 30-80 | <20 |
| (HLB > 12) | | |
| Hydrophilic Cosolvents (ex: PEG, | 0-50 | |
| propylene-glycol, Transcutol ®) | | |
| Betaine as Hydrophilic Co-solvent | — | 80-98 |

These proportions for the new Type V are given for dry forms by excluding water, the presence of which nevertheless remains necessary for the production of the products of the invention.

It should be noted that conventional types IV are subjected to precipitation phenomena when they are resolubilized in water (ionic forces), phenomena at which the products of the invention are remarkably resistant over long periods (test in H₂O).

The present invention describes these new liquid crystals with remarkable properties and which constitute new chemical and/or physical and/or biological entities.

The present invention also describes the novel methods for obtaining these new chemicals and/or biochemical and/or physical entities in the form of liquid crystals and/or nano-emulsions and/or nanocrystals.

The present invention also describes the novel combinations and/or combinations of these liquid crystals and/or new vectors with different soluble and/or insoluble entities, for obtaining new pharmaceutical and/or chemical and/or biochemical and/or physical entities in liquid crystal forms.

These new liquid crystals have remarkable and versatile properties either on their formations, their organizations, their behaviors, their changes in states, their shapes, their properties, as well as in their various industrial applications.

In addition, it has been discovered that the present liquid crystals possess remarkable solubilizations and/or encapsulations properties of pharmaceutical active agents and/or dietary active agents and/or cosmetic active agents, said active agents being poorly, very poorly or not water-soluble and in particular hydrophobic active agents/entities having a Log P greater than 1, preferably a Log P greater than 2, more preferably a Log P greater than 3, even more preferably a Log P greater than 4, advantageously a Log P greater than 5, more advantageously a Log P greater than 6, preferentially a Log P greater than 7, more preferentially a Log P greater than 8, very preferentially a Log P greater than 9.

### DETAILS OF EMBODIMENTS

### Betaine

Betaine, CAS Number: 107-43-7, Linear formula: (CH₃)₃N⁺CH₂COO⁻, Molecular Weight: 117.15 is preferred.

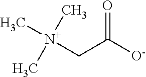

In order to obtain the present liquid crystals, the anhydrous betaine is preferred. The monohydrate form can also be used, alone and/or in combination with the anhydrous form.

### Surfactants & Excipients

In general, each surfactant is assigned a number between 1 and 20, the set constituting the Griffin scale. This number is called HLB index (Hydrophilic/Lipophilic Balance), index 1 is given to oleic acid and index 20 to potassium oleate.

This scale allows to assess the lipophilic characteristics or, on the contrary the hydrophilic characteristics of an amphiphile. For example, an HLB value of less than 9 means that the amphiphile is lipophilic; it will then be used as an anti-foam agent (if HLB<3), as an emulsifier for water in oil (if 3<HLB<6) and as wetting agent beyond. A HLB value superior to 10 means that the amphiphile is hydrophilic it may be used as emulsifier for oil in water (if 9<HLB<13), as detergent (if 13<HLB<15), and as solubilizer beyond 15.

According to the invention, the surfactant and/or the surfactant and/or the surfactant is water-soluble and has an HLB of equal or greater than 10, HLB being the “hydrophilic/hydrophobic balance”.

According to a particular aspect of the invention, the water-soluble surfactant having an HLB equal or greater than 10 can also be chosen from the groups consisting of esters of PEG-6 of almond oil, esters of PEG-60 almond oil, PEG-6 esters of apricot kernel oil, PEG-4 ester capric triglycerides, PEG-4 caprylic/capric triglyceride complexes, caprylic/capric glycerides/PEG-6 esters, caprylic/capric glycerides/PEG-8 esters PEG-50 castor oil esters, PEG-5 hydrogenated esters, PEG-7 castor oil hydrogens esters, 9 PEG-9 hydrogenated castor esters, PEG-6 maize oil esters, PEG-8 ester corn oil, PEG-60 ester corn glycerides, PEG-6 olive oil esters, palm kernel oil/PEG-6 ester palm oil, PEG-6 palm kernel oil/PEG-6 ester oil esters with palm kernel oil and PEG-6 and palm oil, PEG-40 palm kernel oil, PEG-6 peanut oil esters, saturated C8-C18 fatty acid glycerol esters, glyceryl esters of saturated C12 to C18 fatty acids, glyceryl laurate/PEG-32 laurate, glyceryl laurate, glyceryl/PEG 20 laurate, glyceryllaurylglyceryl/PEG 32 laurate, glyceryl, lauryl glyceryl/PEG 40 laurate, glyceryloleate/PEG-20 glyceryl, glyceryloleate/PEG-30 oleate, glycerylpalmitostearate/PEG-20 glyceryl, PEG-32, glyceryl ethylate/PEG-32 stearate/PEG-32 stearate, saturated polyglycolized glyceridesPEG-6 triisostearin esters, PEG-6 triolein esters, PEG-25 trioleate esters, polyoxyl 35 castor oil, polyoxyl 40 hydrogenated castor oil, polyoxyl 60 hydrogenated castor oil, PEG-8 caprate PEG-8, PEG-8 caprylate, PEG-8 caprate PEG-8 laurate, PEG-8 oleate, PEG-8 stearate, PEG-9 caproate, PEG-9 caprylate, PEG-9 caprate PEG-9 laurate, PEG-9 oleate, PEG-9 stearate, PEG-10 caproate, PEG-10 caprylate, PEG-10 caprate PEG-10 laurate, PEG-10 oleate, PEG-10 stearate, PEG-10 laurate PEG-12 oleate, PEG-15 oleate, PEG-15 oleate, PEG-10 20 laurate, PEG-20 oleate, caprylic/capric glycerides, caprylate/caprate diglycerides, glyceryl monooleate, glyceryl ricinoleate, glyceryl laurate, glyceryl dilaurate, glycerol dioleate, glyceryl mono/dioleate, glyceryl caprylate/caprate, mono- and diglycerides with a C8/C10 medium chain, mono- and diacetylated monoglycerides, polyglyceryl, polyglycerol oleate, polyglyceryl-2 dioleate, polyglyceryl-10, polyglyceryl-10 mono dioleate, propylene glycol caprylate/caprate, propylene glycol dicaprylate/dicaprate, propylene glycol monolaurate, propylene glycol distearate, sorbitan monostearate glycol dioxide, sorbitan monostearate PEG-20, sorbitan monooleate PEG-20, poloxamers such as Pluronic, Kolliphore, Synperonic, poloxamer 108, poloxamer 124, poloxamer 182, poloxamer 238, poloxamer, poloxamer 407, sorbitan monolauratesorbitan monopalmitate, sorbitan monoleate, sorbitan monostearate, sorbitan tristearate, d-alpha-tocopherylpolyethylene glycol 1000 succinate, polysorbate 20, polysorbate 40, polysorbate 60, polysorbate 65, polysorbate 80, polyethylene glycol 660 12-hydroxystearate; polyglycolized glycerides, polyoxyethylene glycerides, polyethylene glycol esters, fatty acid esters of polyethylene glycol glycerol, transesterification products of oils and alcohols, polyglycerol fatty acids, glycerol fatty acid esters, polyglycerol fatty acid esters, propylene glycol fatty acid esters, mono and diglycerides, fatty acid esters of polyethylene glycol sorbitan, polyoxyethylene polyoxypropylene block, sorbitan fatty acid esters, d-alpha-Tocopheryl Polyethylene Succinate 1000, Polyoxyethylene Glycol 12-Hydroxystearate 660, Polysorbates and mixtures thereof.

The water-soluble surfactants having an HLB of equal or greater than 12 of the above lists being according to the invention preferred.

In an advantageous and preferred aspect of the invention, the polysorbates or Tween 20 (E432-HLB 15), Tween 80 (E433-HLB 15), Tween 40 (E434-HLB 15.6), Tween 60 (E435-HLB 14.9), Tween 65 (E436-HLB 10.5), as well as their mixtures, will be preferred due in particular to their low toxicity, their low costs, their ease of use, their effectiveness, as well as their uses at ambient temperature (15 to 25° C., or even 30° C. and more) for the realizations of the liquid crystals and/or for the realizations of the compositions comprising the liquid crystals of the invention and/or the liquid crystals of the invention comprising cargoes entities and/or host entities and/or active hydrophobic entities.

According to another advantageous aspect of the invention, the plant saponins generally and particularly the extracts of the Quillaja Saponaria Molina (E999) plant and/or QS-21 a highly purified saponin thereof can be used as surfactants as a replacement for polysorbates and/or other surfactants, and/or used in combinations with polysorbates and/or other surfactants.

The extract Quillaja Saponaria Molina typically contains molecules of non-ionic surfactant types which can reorganize into micelles having a significant emulsification power. In the context of the invention, they may advantageously replace and/or supplement the polysorbates (s) in formulations and/or products claiming compositions 100% of vegetal (and/or plant) origins and/or Vegan products; and/or products claiming a better acceptability and/or tolerability by the users.

Very advantageously, the present liquid crystals as well as their various industrial applications, require, due to the use of betaine, minimal or much smaller amounts of surfactants such as polysorbates. In this context, it is possible to speak of synergistic activities of betaine and/or of liquid crystals of betaine with the one or more surfactant(s).

It is established that these synergistic activities, as well as the properties (efficacy/safety, solubilizations, etc.) of the present liquid crystals are obtained only in the particular betaine/surfactant/solvent proportions described herein.

### Solvents

According to the invention, the preferred solvent is water for obtaining the present liquid crystals.

Nevertheless, when the present liquid crystals are used to solubilize certain hydrophobic molecules and/or entities, it may prove to be useful and/or necessary and/or desirable during the procedure for obtaining, to solubilize said hydrophobic molecules and/or entities in a water-miscible organic solvent, such as ethanol and/or methanol and/or propanol and/or butanol and/or DMSO and/or chloroform and/or similar acceptable water-soluble solvents.

In general, any water-miscible solvent also making it possible to solubilize the cargo/host entity can be used; so that this solvent can be removed, preferably entirely eliminated, in a subsequent step of producing the final product, in order to obtain a dry and/or substantially dry product free and/or substantially free of this solvent; and/or liquid product and/or a product able to be in a liquid form, said products substantially free of this solvent.

The one or more acceptable solvent(s) may also be retained for the liquid and/or semi-liquid formulations and/or in forms of gels.

Nevertheless, according to the invention, the final dry forms, i.e., the forms free and/or substantially free of liquids and/or of liquid solvents (other than the one or more surfactants of the invention) will be preferred. These dry final forms preferably being in powdered forms, powders, micro-powders (micronized), nano-powders (particles having diameters of less than 1 μm, 0.5 μm or 0.1 μm), chips, granules, micro-granules; and combinations thereof.

According to the invention, the dry forms have increased stability properties.

### Methods of Preparation

According to the invention, glycine betaine liquid crystals may form and/or organize beyond the critical micelle concentration (CMC) of the betaine and in the presence of a precise amount of one or more surfactants and/or one or more nonionic surfactants having an HLB equal or greater than 12, preferably greater than 13, preferably greater than 14.

The mixtures are preferably carried out in the strict proportions of the invention, the preferred solvent being water.

It is important to note that the too low concentrations of surfactants, as well as the too high concentrations of surfactants give poor or no results at all with, in particular, in case of high concentrations of surfactants the bleaching of the solutions, with particle sizes exceeding 500 nanometers. On the contrary, too low concentrations of surfactants do not allow the dissolution of the cargo substances.

The precise proportions of betaine/surfactant, as well as the proportions of the solvent(s), being imperative for the realization of the invention.

The successful formulations give rise to transparent solutions having integrated (solubilized) the cargo entities, and having particle sizes below 250 nanometers. This threshold, with the quantity of solubilized cargo determine the success or not of the formulations.

These conditions must be combined in the proportions of the invention in order to obtain the liquid crystals of the invention. They are also necessary for the expression and/or acquisition of the remarkable properties of the present liquid crystals and/or nano-emulsions, as regards their solubilizers of insoluble or very poorly soluble entities.

It should be noted that this drastic reduction in the quantity of surfactants in the present products goes against the state of the art which specifies, on the contrary, high amounts of surfactants (see Table 1 for example). A notable improvement with respect to the state of the art is that the present liquid crystals make it possible to obtain dry products and/or substantially dry products with highly improved stability profiles.

Contrarily to the state-of-the-art liquid products, present products being in dry forms have an improved stability vs. such known liquid products.

In the other hand, for reaching dry products by the formulations of the prior art is possible only after their mixing with zeolites and other amorphous substances (e.g., silica) in order to allow a dry physical appearance. This dry appearance would be impossible to reach otherwise, due to the high concentrations of surfactant (used in the previous art) which are then in semi-liquid (viscous) state and thus cannot be dried.

For example, when referring to Table 1 LFCS, Types I, II, IIa & IIb contain all oils which are impossible to be dried. Type IV products are free of oils, but contain water-soluble surfactants (HLB>12) in a proportion of 30 to 80% which are impossible to dry; without speaking of the other ingredients such as water-insoluble surfactants (0 to 20%) and hydrophilic co-solvents such as PEG, propylene glycol or Transcutol® (0 to 50%) which are also impossible to dry.

Conversely, the present products use only water-soluble surfactants (HLB>12) in a typical proportion of 2 to 5% (water & cargoes not included) and 95% to 98% of betaine as a co-solvent.

According to invention embodiments, proportions of water-soluble surfactants (HLB>12) of less than 2%, such as 1.5%, 1% or 0.5% are preferred, betaine amounts being accordingly increased to 98.5%, 99% or 99.5% to reach the 100% total ingredients. (not taking into account the possible hydrophobic entity and water present in the liquid crystal)

The products in dry forms are obtained in a crystallization step after evaporation of the water and of the optional other solvent in addition. Since the cargoes entities are enveloped, their lipophilic characteristics cannot be expressed and do not enter into account in the drying processes.

Consequently, the advantages of the present dry and/or substantially dry products are obvious and undeniable vs. the products known in the art, both with respect to the ease of industrial implementation, costs, stability of the dosage forms, the increased bioavailability of the hydrophobic cargoes entities, the increased effectiveness of the cargoes entities, the very low toxicity of the betaine (which is an endogenous compound), and the absence of allergic reactions (vs. for example PEGs), and the combinations of the preceding advantages.

As regards the formation of the present betaine liquid crystals alone (without hydrophobic cargo(es)), usual proportions by weight concerning the betaine ratio and the water and/or the solvent ratios, are from 0.5 to 1.5 (ratios from 1 to 3) to 1.5 to 0.5 (ratios from 3 to 1) respectively, and from 0.2 to 0.001 by weight for the surfactant, the number (amount) 2 representing the 100% of the mixture.

The usual proportions of the ingredients, for the formation of the present liquid crystals and/or nanoemulsions and/or vectors and/or transporters, are in percentage by weight between 25 and 75% of betaine, 25 and 75% of water and/or solvent and between 0.1% and 10% of surfactant (s), to which may be added, if desired, the host/cargo entity to be solubilized/encapsulated/transported.

For example, a usual mixture according to the invention consists of 2 grams of betaine (2 parts by weight), 2 milliliters of water (2 gr or 2 parts by weight) and 50 to 100 milligrams (0.05 to 0.1 part by weight) of surfactant having an HLB greater than 12, to which one or more insoluble entities and/or cargoes may be added.

The usual proportions of the cargoes and/or host entities according to the invention are situated as a percentage of weight in their mixtures with the liquid crystals between 0.1% and 20%. The total percentage betaine/solvent (s)/surfactant (s)/cargo entity (s) is 100%. These proportions are applied to the liquid forms of the present crystals.

By way of example, but only illustrative and in no way limiting, in order to form the present liquid crystals, with optionally their cargoes could be composed as follows: 2 grams of anhydrous betaine, 2 grams of water, 50 to 100 mg of surfactant and 100 mg to 200 mg of cargo/host entity, the mixture operating at room temperature (between 15° C. and 30° C.) and at a normal atmospheric pressure of 1 atm (1013.25 hPa).

According to the invention, the solubilizations processes are advantageously carried out at ambient temperature and under normal atmospheric pressure. However, if the profile of the host/cargo entity requires it, it is possible to vary the atmospheric conditions by a factor of 1 to 1000, according to the particular needs of the invention.

According to a particular aspect of the invention, the weight of the cargo entity may preferably be greater than that of the surfactant.

According to the invention, the order and the sequences of mixtures and/or solubilizers of the ingredients, as well as the respective proportions of said ingredients, are important since they condition the quality and the properties of the liquid crystals obtained. Varying these parameters can lead to results ranging from not at all effects (not forming the liquid crystals of the invention) to 100% of solubilizations and/or encapsulations effects.

For example, it has been noted, in some cases, that the solubilization of the surfactant in the solvent first, then the addition of the betaine and/or the addition of the cargo entity (s) could give better results as regards the solubilization and/or encapsulation and/or association of said cargoes entities.

In other cases, it has been found that the dry mixture of the betaine powder with the surfactant, then, if desired, the cargo entity, then the solubilization in the (water) or the solvents gives better results.

Likewise, the mixing conditions are important, in particular by way of example but without being limited thereto, the mixtures by centrifugation and/or mixtures by ultrasound and/or mixtures by cavitation and/or by shear forces and/or mixtures by pressure and/or mixtures by microfluidisation and/or mixtures under particular thermal conditions, and combinations thereof.

The combinations, for example, of alternating centrifugations with exposures at low temperatures (below 5° C.) make it possible to optimize the methods of the invention and/or to obtain, according to the order of the steps of the specific products.

These conditions can influence and/or generate and/or organize and/or induce and/or condition the director of the present liquid crystals. The director being the vector indicating the average direction of alignment and/or organization and/or orientation of the molecules of the present liquid crystals.

According to the invention, the director of the present liquid crystals can be induced and/or influenced and/or generated and/or directed by the mechanical and/or thermodynamic and/or shear and/or chemical and/or electrical and/or optical and/or magnetic forces, as well as their combinations and/or their alternations.

According to an aspect of the invention, the centrifugal processes (500 to 10,000 rpm corresponding to 500 to 10,000×g) and/or ultracentrifugations (10,000 to 100,000 rpm corresponding to 10,000 to 100,000×g) being preferred, alone or in combinations with other means of inductions (cited above), both for their efficiencies for obtaining the present liquid crystals and by the ease of their use.

According to an advantageous aspect of the invention, ultracentrifuge processes may be used and may reach 250,000 g, 500,000 g, 750,000 g or even more for the needs of realizing the invention.

According to the invention, these methods of centrifuging and/or ultracentrifugations and/or cavitations and/or shears, in addition to generating/conditioning the directors of present liquid crystals, prove to be particularly useful when it involves integrating and/or trapping and/or loading and/or housing and/or associating and/or encapsulating and/or solubilizing the cargoes entities described in the invention.

The procedures of embodiments for making the present crystals can also be carried out at ambient temperatures (15 to 25° C., or even 30° C. and more).

The present invention can be carried out at room temperature, it is particularly suitable for the development of vaccines and/or products and/or molecules and/or proteins and/or fragments of the living being, having sensitivity and/or lack of stability and/or denaturation when exposed to heat and/or to caloric excess.

According to the invention, the liquid crystals associated with their cargoes and/or the resulting products are able to be subjected and/or being subjected to one or more solubilizations processes and/or to one or more drying processes and/or to one or more heating processes and/or to one or more dehydration processes and/or to one or more freeze-drying processes and/or to one or more atomization processes and/or to one or more precipitation processes and/or to one or more drying processes and/or several sedimentation methods and/or one or more gravitation methods and/or one or more cryopreservation methods and/or one or more irradiation methods and/or one or more separation methods and/or one or more skimming methods and/or one or more sonication methods and/or one or more ultrasonic methods and/or one or more trituration methods and/or one or more mixing methods and/or one or more methods of magnetic mixture and/or one or more acoustic mixing methods and/or one or more ultrasound mixing methods and/or one or more microfluidisation methods and/or one or more solubilization methods and/or one or more dissolution methods and/or one or more molding methods and/or one or more compression methods and/or one or more depression methods and/or one or more desiccation methods and/or one or more fractional solidifications methods/or one or more crystallization methods and/or one or more germination methods and/or one or more sublimation methods and/or one or more filtration methods and/or one or more ultrafiltration methods and/or one or more osmosis methods and/or one or more retention methods and/or one or more centrifugation methods and/or one or more ultracentrifugation methods and/or one or more methods of retention and/or one or more methods of centrifugation and/or one or more methods of ultracentrifugation and/or several spray drying processes and/or to one or more concentration processes and/or to one or more evaporation processes and/or to one or more freezing processes; for obtaining the liquid crystals of the invention and/or of a product and/or of a dry product, said dry product being free and/or substantially free of water and/or solvent.

According to invention embodiments, the cargo entities may be grinded with means known from the Art such as ball grinding and/or ball planetary grinding to sizes below 1 micrometer (<1 μm) before being mixed to the other ingredients (surfactant(s) and betaine), the mixtures being further solubilised in water to form the liquid crystals of the invention.

According to embodiments, grinding/milling can reach cargo entities particles sizes having diameters inferior to 1 μm, preferably inferior to 0.5 μm, advantageously inferior to 0.25 μm, preferentially inferior to 0.1 μm for the obtaining of amorphous state of present cargo entities.

The nano-grinding of the cargo entities under the micrometer size allows that the cargo entities to be in an amorphous state. Amorphous materials exhibit distinct physicochemical properties compared to their respective crystalline counterparts.

Amorphous (cargo) entities are markedly more soluble than their crystalline counterparts.

This increased solubility is a way for increasing bioavailability of poorly water-soluble entities.

Accordingly, present betaine liquid crystals provide high specific surface areas, consequently increasing the dissolution rate and solubility of present cargo entities. However, maintaining the amorphous state in finished galenic products is a challenge due to amorphous material tendency to return to its crystalline (insoluble) state.

According to important embodiment of the invention, present betaine liquid crystals freeze/fix/maintain the cargo entities in their amorphous state, which leads to improved solubilities and/or dissolutions of said cargo entities within the finished galenic products.

Accordingly, present betaine liquid crystals are claimed to maintain at least 70%, preferably at least 80%, advantageously at least 90%, preferentially at least 95% the present cargo entities in their amorphous state within the finished galenic products, until their utilizations by a subject in need.

### Particular Properties of Present Liquid Crystals

According to the invention, the present liquid betaine crystals have birefringence properties.

Iridescence and birefringence are the established hallmarks of nanoformulations.

According to the invention, the present liquid betaine crystals have the characteristic features of nanoemulsion with their optical clarity, clear or bluish tint appearance and small particles size (20-200 nm) which makes them insensitive to gravitational instability, dilution and temperature.

Above of all, present invention liquid crystals while achieving said properties using lower surfactant concentration and by supplying external energy differentiate them from microemulsion, which uses higher amount of surfactant thereby making them toxic for human body.

According to the invention, the present liquid betaine crystals have amphitropic properties, that is to say can exhibit thermotropic properties (be dependent on temperature) and/or lyotropic properties (be dependent on the concentration and/or temperature).

According to the invention, the mesophases of the liquid crystals of betaine are organized and/or have an internal interfacial zone constituted by hydrophilic and hydrophobic domains. These domains are distinct, separated and capable of trapping hydrophilic and/or hydrophobic and/or amphiphilic entities.

The nanoscale porosity of the present liquid betaine crystals provides a large internal and external surface in which, according to the present invention, they are housed and/or inserted and/or associated with the hydrophobic and/or hydrophilic and/or amphiphilic entities and/or active agents of interest. It is possible to speak “of molecular sponges”.

According to one aspect of the invention, the present liquid crystals may be ordered in a hexagonal columnar phase, where the betaine molecules and the surfactant (s) (HLB>12) form long cylinders which are stored in a hexagonal network. It can be said that the present liquid crystals form molecular channels, said channels being capable of encapsulating and/or transporting and/or associating with other entities, in particular entities having a Log P greater than 2. In this case, the cargoes and/or companion entities would constitute the stuffing of said cannelloni.

According to another aspect of the invention, the present liquid crystals may be ordered in lamellar phase, where the betaine molecules and the surfactant (s) (HLB>12) form long sheets separated by thin layers of water. It can be said that the present liquid crystals form molecular pasticcios, said pasticcios being able to encapsulate and/or transport and/or to associate with other entities, in particular entities having a Log P greater than 2. In this case, the cargoes and/or companion entities would constitute the farce of said pasticcios.

According to another aspect of the invention, the present liquid crystals may be ordered in cubic phase (also called viscous isotropic), where the betaine molecules and the surfactant (s) (HLB>12) form spheres creating a dense cubic lattice. These spheres may also be connected to one another, forming a bi-continuous cubic phase.

According to the invention, the present liquid crystals are of hexagonal phase (hexagonal cylindrical phase) and/or of lamellar phase and/or of bi-continuous cubic phase as a function of the concentration of betaine and/or as a function of the betaine/surfactant ratio and/or of the betaine/solvent ratio.

Under certain conditions, the present liquid crystals may also be ordered in an inverted hexagonal columnar phase and/or a reverse cubic phase reverse cubic phase as a function of the concentration of betaine and/or as a function of the betaine/surfactant ratio and/or of the betaine/solvent ratio.

According to a particular aspect of the invention, high concentrations may generate reverse phases such as reverse hexagonal columnar phases (water columns encapsulated by betaine+surfactants) and/or reverse micellar phases (liquid crystals with spherical water cavities).

In addition, according to the invention, each crystal obtained can be characterized by one or more measurements selected from the group of: water solubility of (mg/ml), a size of, density, diffraction, refraction, reflectivity, photo-reactivity, polarization, wavelength, viscosity, conductivity, solubility, microscopy, resistivity, structure, spectroscopy, mass spectroscopy, Raman spectroscopy, DLS, NMR, HPLC, XRPD (X ray powder diffraction), melting point, cloud point, cargo, cargo %, hygroscopicity volumetric weight, isoelectric point, Kraft point, displacement of Stockes, calorimetry, microcalorimetry as well as any other measurements known by the state of the art and used to characterize a crystal and/or a nanocrystal.

Depending on the hydrophobic/solubilized hydrophobic substance and its profile, each liquid crystal and/or each hydrophobic substance/liquid crystal association, described herein, will have a unique profile according to the invention and may be considered as a new chemical and/or physical and/or therapeutic entity.

The modular and versatile side of the present liquid crystals constitutes a real advantage, due to the simplicity of their realizations as well as to their various industrial applications.

Similarly, the robustness and simplicity of their embodiments constitute an economic and/or technical advantage compared with the complicated procedures of the prior art regarding the solubilization of entities that are not, or poorly, water-soluble.

The procedures of embodiments for making the present crystals can be carried out at ambient temperatures (15 to 30° C.). likewise, the procedures of drying and/or removal procedures of the solvent (s) can be conducted below 70° C. preferably at temperatures not denaturing the cargoes (proteins, peptides, RNA, DNA, aptamers, virus envelopes and/or bacteria, biological entities, living fragments, etc.), which constitutes an advantage.

The liquid crystals of the invention, in addition to their solubilizing power, may also offer the host/cargo entities a protection against the attacks tending to denature them. These protective activities, on the cargo entities mentioned above, are also useful against physical and/or chemical and/or biological and/or enzymatic and/or luminous (photooxidation) denaturants other than temperature.

Finally, the very low toxicity of the ingredients, in particular betaine, as well as the significant reduction of surfactants compared to the prior art, elect the present liquid crystals as better alternative to all known pharmaceutical and/or dietetic and/or cosmetic formulations which use PEG or other solubilizers. The present liquid crystals can advantageously replace most of the formulations of the prior art. These differences, visible to the naked eye can be observed concerning in particular the birefringent nature of the successful combinations, their transparency, their opalescence, their color changes, their light diffusions, their viscosities, their surface tensions, their crystallization profiles, or quite simply the capacity of the present liquid crystals to solubilize various entities, in particular hydrophobic entities, all these results being visible macroscopically.

According to the invention, the present products form nanostructures in solution and that these stable nanostructures make it possible in addition to solubilize the host entities, to improve their dissolution profiles in water, and/or in physiological liquids.

Similarly, for the more water-soluble entities having a Log P of less than 1.5 but greater than 1, the present liquid crystals offer encapsulations properties and/or combinations that make it possible to optimize the bioavailability and/or the stability and/or acceptability and/or safety and/or efficacy profiles of these entities. Stability in the present context means the protection that these crystals supply against degradations triggered by agents and/or processes such as oxidation and/or light and/or UV and/or acids and/or bases and/or enzymes and/or any biological and/or chemical and/or physical phenomenon that would deteriorate and/or denature the cargoes entities before they are delivered to the target sites (cells and/or tissues and/or organs).

### Drying & Germinations

According to the invention, the present crystals are suitable and/or capable of being subjected to the reduction and/or elimination of their solvents in order to initiate a preferably spontaneous nucleation (or germination) which corresponds to the appearance of a stable crystalline phase obtained from liquids in superfusions and/or supersaturated solutions.

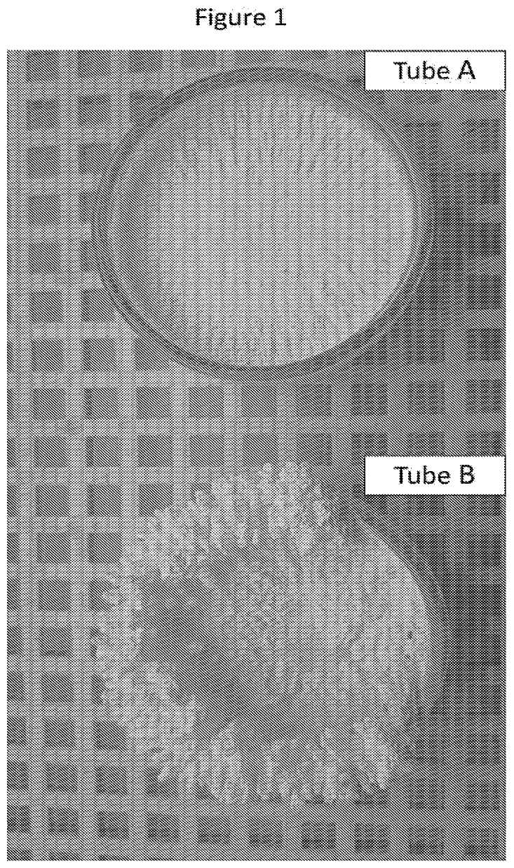

Since this nucleation is of the heterogeneous primary type with germs forming on the walls of the container, in opposition to the homogeneous primary nucleation of the solutions of betaine alone where the seeds remain confined in the volume of the solution as can be seen in FIG. 1.

According to the Invention, the inventive Betaine Liquid Crystal(s) have nucleation of heterogeneous primary type as can be clearly seen in FIG. 1 Tube B comparatively to the homogenous nucleation of Tube A solution (Betaine control aqueous solution).

According to invention embodiments, this heterogenous nucleation, which translate in the new betaine liquid crystal ability to solubilize cargoes entities in water and/or to protect cargoes entities, can only be reached when combining the betaine of the invention (CAS Number: 107-43-7) with one or more surfactant having HLB>12 (preferably Polysorbates and/or Quillaja Saponaria) in the exact proportions described in the present disclosure.

From a thermodynamic point of view, the heterogeneous nucleation requires much less energy than the homogeneous nucleation.

In embodiments, industrial processes for obtaining the present crystals which use glass containers and/or glass walls and/or glass crystallizers are preferred and claimed in order to induce and/or to optimize this heterogeneous nucleation.

Surprisingly, the betaine/surfactants (with HLB>12) combinations in the proportions of the invention induce new heterogeneous nucleation.

These nucleating agents are particularly useful for the syntheses and/or crystallizations and/or the methods of concentrations of forms containing proteins and/or fragments and/or whole parts derived from the living being (insulins, viruses, DNA, RNA, aptamers, enzymes, etc.). they allow the same and/or similar ways of concentrating and/or transporting the other cargo entities of the invention.

According to one aspect of the invention, the present crystals have a crystallization governed by the topology of the container. Under particular conditions, the present crystals may also self-assemble and/or self-crystallize at ambient temperatures.

According to one aspect of the invention, the present cargoes entities (described below) promote and/or optimize the formations of these heterogeneous nucleations.

According to a particular aspect of the invention, the present liquid crystals transform the cargoes entities into their most stable and most soluble polymorphs, preferably cargoes entities are in amorphous states within the betaine liquid crystals of the invention. This is entirely unexpected in view of the prior art. (Nucleation and phase transitions in pharmaceutical chemistry-Laurent Lafirmer-Thesis September 2002).

Unexpectedly, these nucleation/crystallization processes take place with crystal growth (FIG. 1 Tube B) completely different from those of the betaine alone in isotopic phase, simply solubilized in water in the same proportions (FIG. 1 Tube A). Indeed, both at their growth kinetics and their amplitudes and their growth rates, that is to say the increase in size of the seeds in order to lead to the crystals of the invention differ very significantly from the solutions of betaine alone, i.e., the betaine not associated with the surfactants having an HLB equal or greater than 12, as shown in FIG. 1.

The growth of the present crystals shows to be of coral type with columns and/or concretions which grow by stacking on one another; this type of structures may be fractal representations of the molecular structures of the present liquid crystals.

These new betaine crystals have remarkable and new physical/chemical and/or optical properties described by the present application.

The crystals of the invention show velocities, growth amplitudes, crystalline volumes, original densities, as well as macroscopic and/or microscopic and/or new molecular forms.

Unexpectedly, the liquid crystals of the invention transform upon drying into crystals having a particular growth factor.

In one aspect, the liquid crystals of the invention have, in certain betaine/solvent/surfactant proportions, a cloud point which is temperature dependent.

The invention also describes a method for forming liquid crystals by nucleation (or germination) corresponding to the appearance of a stable crystalline phase from a supercooling liquid and/or from a solution supersaturated in betaine. The process continues with the crystal growth, that is to say the increase in size of the seeds in order to lead to the crystals. It is characterized by the stacking on the surface of the crystal of new particles which are housed in preferential sites.

According to the invention, the 2 germination models of the crystals derived from supersaturated solutions can be applied:

The so-called classical model, where the growth is promoted by the formation energy of the crystal, while the dissolution is promoted by the surface tension of the interface created between the crystalline cluster and its liquid environment.

The two effects are counterbalanced for a particular cluster size, called “critical size”. Consequently, the crystalline clusters smaller than the critical size tend to dissolve under the effect of thermal fluctuations; conversely, the crystalline clusters larger than the critical size tend to grow on average, until macroscopic crystals are formed.

The second model, referred to as two-step model, applies for the crystallization of the proteins and is based on the formation of a crystalline cluster (cluster) containing a solution of the species to be crystallized, followed by the orderly rearrangement of these species to give a seed which, like the conventional coarse model, to give a crystal.

Unexpectedly, the drying solutions transform into crystals having a particular growth factor, that is to say a growth substantially different from that, possible, of its ingredients, in particular the betaine alone in solution.

It noticeable that when used outside the proportions of the invention the surfactants of the invention do not crystallize or are hardly or very difficult to dry, which may induce viscous and/or tacky final products with little or no pulverulent propensity.

Unexpectedly, the solutions from which these crystals originate make it possible to solubilize and/or increase the bioavailability and/or increase the half-life and/or increase the efficiency and/or increase the safety and/or be physiological vectors and/or constitute stealth vectors and/or protective envelopes for different molecules and/or compounds and/or entities that are poorly soluble or not soluble in water.

According to the invention, the germinating/nucleating methods make it possible to optimize the quality and/or the proportion and/or the amount and/or the content of the cargo ingredients in the final composition and/or in particular to reduce, preferably significantly, the amounts of surfactants (HLB>12) used.

The reduction in surfactants quantity is advantageous and desirable for the safety and the acceptability of the final products by the living being.

According to the invention, the present liquid crystals are able to be subjected to one or more crystallizations to obtain a powder and/or a dry product and/or a salt and/or crystals, free and/or substantially free of water and/or free of solvents.

The invention also describes the nucleation or germination of the present liquid crystals in order to obtain a crystal and/or a water-soluble crystalline form. According to the invention, this growing crystallization surprisingly makes it possible to optimize the solubility properties and/or the stability of the cargo entities and/or the combinations cargo entities of the invention.

According to a particularly advantageous aspect of the invention, the drying makes it possible to “freeze” (block/hold/stabilize/keep in a fixed state) the structures and/or nanostructures obtained until administration. The stability of the compositions of the invention are therefore increased compared with those of the existing formulations.

The drying methods used according to the invention may all be known in the prior art and may for example be methods using heat and/or cold and/or air and/or pressure and/or shear and/or any method known by those skilled in the art making it possible to eliminate or substantially eliminate one or more liquid solvents. The drying methods must preferably comply with the formation of the crystals and/or the quality of the cargoes entities by being “softs” methods, that is to say maintaining and/or optimizing the properties of the ingredients and/or cargoes and/or combinations.

According to an advantageous aspect of the invention, the drying operations can be carried out at temperatures of less than 100° C., preferably less than 90° C., more preferably less than 80° C., preferably less than 70° C., preferably less than 60° C., more preferably less than 50° C., advantageously less than 40° C., preferentially less than 30° C., even more preferentially less than 20° C., very preferentially less than 10° C.

The drying methods according to the invention may all be known in the prior art and may for example be methods using heat and/or cold and/or shear and/or air and/or pressure.

The present dry and/or substantially dry products obtained having a moisture content and/or a content of liquid content of less than 10%, preferably less than 9%, more preferably less than 8%, preferably less than 7%, preferably less than 6%, more preferably less than 4%, advantageously less than 2%, preferentially less than 1%, even more preferentially less than 0.5%, very preferentially less than 0.1%.

The present pulverulent products and/or dry and/or substantially dry products being stable and able to be resolubilized in one or more solvents, preferably water, and/or one or more physiological liquids.

The present products and/or methods having frozen and/or substantially fixed and/or time-stabilized dissolution and/or solubilization properties.

The present pulverulent and/or dry and/or substantially dry products having stability such that when they are resolubilized in water over a period of 1 to 36 months, the solubility and/or the content of the API (host entity) and/or the particle size and/or the zeta potential and/or the polydispersity of the particles in the solutions obtained are substantially similar to those of the initial solution with a variation of less than 20%, preferably less than 15% more preferably less than 10%, preferably less than 8%, preferably less than 6%, more preferably less than 4%, advantageously less than 3%, preferentially less than 2%, even more preferentially less than 1%, very preferentially less than 0.5%.

The solutions from which these crystals originate make it possible to solubilize and/or increase the bioavailability and/or increase the half-life and/or increase the stability and/or increase the efficiency and/or increase the safety and/or be physiological vectors and/or to be stealth vectors and/or to be protective envelopes for different molecules and/or compounds and/or entities soluble and/or poorly soluble and/or not soluble in water.

The advantage of the invention lies in obtaining stable pulverulent products, preferably including cargoes entities, the solubility properties of which, acquired by virtue of the present liquid crystals, are frozen (blocked/held/stabilized) by means of one or more drying and/or dehydration, said stable products being able to be reconstituted and/or resolubilized on demand in a liquid, preferably in a physiological liquid and capable of retaining their properties of solubility giving them a better bioavailability after administrations.

According to an advantageous aspect of the invention, the present liquid crystals are stable in acidic environments (such as the stomach) and are also stable in basic environments such as the intestinal environment where the absorption takes place towards the blood flow. The combination of this stability at the various pH values (1 to 8), as well as their ability to protect and transport cargoes entities, makes present crystals of the choice candidates as regards the various formulations, in particular oral formulations of the invention. By way of example, the present crystals are optimal transporters for insulins and/or other proteins in oral formulations and/or in formulations known in the art.

The invention also describes the use of surfactants at cold temperatures, which proves to be a simpler method with much less surfactants and able to provide final products in dry forms.

Indeed, it has been noted that the excess surfactant contrary to the other formulations of the art gives poor results. Unexpectedly, the low concentrations give good results as to the formations and/or to the properties of the present liquid crystals.

According to an advantageous aspect of the invention, the present betaine liquid crystals require amounts of surfactants substantially less than the amounts required in the prior art for solubilizing and/or for protecting a given quantity of one or more cargo entities listed in the present application; said amount of surfactants, when using betaine liquid crystals can be reduced by 10%, preferably by 20%, more preferably by 30%, even more preferably by 40%, advantageously by 50%, more advantageously by 60%, even more advantageously by 65%, very advantageously by 70%, preferentially by 80%, more preferentially by 85%, even more preferentially by 90%, very preferentially by 95%, comparatively to the amounts needed by the prior art for solubilizing and/or protecting the same quantities of the cargo entities listed in the present application.

For example, if in a conventional formulation of the previous art, a quantity of 100 mg of a cargo entity needs for being solubilized (and/or for being protected) 500 mg of surfactants with HLB>12, the present betaine liquid crystals allow identical results with only the use of 50 mg of surfactants with HLB>12 (90% reduction) or even with only the use of 25 mg of surfactants with HLB>12 (95% reduction).

According to the invention, these drying methods also make it possible to freeze (hold in a fixed state) and/or stabilize one or more of the properties of the present liquid crystals.

The advantage of the invention lies in obtaining stable products, that is to say the solubility properties of which are frozen, capable of being reconstituted in one or more liquids, preferably physiological liquids, and capable of retaining their best performance (solubility, stability, bioavailability, acceptability, etc.). According to the invention, an amorphous precipitation can also be obtained, where the cargo entity precipitates in amorphous form in the liquid crystals. The high state of the cargo entity in this system produces much larger dissolution rates than the inert forms of the cargo entities. The conversion of a cargo entity into an amorphous form following the co-precipitation in the crystals of the invention leads to an increased dissolution.

According to the preceding paragraph, the cargo entity acquires an amorphous structure within the liquid crystals of the invention, and is able to remain amorphous in a suitable galenic envelope.

According to invention embodiments, the cargo entity may be transformed in its amorphous state before being mixed with the other ingredients, namely betaine, one or more surfactant with HLB>12 and one or more physiological solvent, preferably water.

The transformation of the cargo entity from its crystallite state in its amorphous state may be realized by grindings and/or mixings and/or dispersings using energies such as mechanical and/or magnetic and/or sonication and/or pression and/or shear. According to invention embodiments, the use of cargo entities in amorphous state may facilitate and/or optimize the realization of present solubilizing/protecting betaine liquid crystals. It may be particularly advantageous in regard of the lower energy required during industrial processing for the obtention of the products of the invention.

One of the concepts of the present invention is to obtain forms and/or crystals and/or betaine crystals with remarkable properties and the examples as well as the emulsifiers (surfactants) and co-solutes described here can be extended to all those known by those skilled in the art, without being outside the field or outside the concepts described by the present invention.

The present liquid crystals and/or their crystallized dry forms are used to increase the water solubility of a hydrophobic substance by a factor of 5, preferably by a factor of more than 10, more preferably more than 50, preferably more than 100, more preferably more than 500, more preferably more than 1000, advantageously more than 2000, preferentially more than 3000, even more preferentially more than 4000, very preferentially by a factor of more than 5000, said solubility being able to be calculated for example by the USP Paddle method and/or by the dissolved weight of the hydrophobic substance in a given volume of water such as mg/ml (milligram/milliliter) or Mole/Liter (examples of descriptive and non-limiting techniques).

The present co-precipitates and/or liquid crystals can be dried one or several times, optionally under vacuum, in order to eliminate and/or substantially eliminate any solvent and/or surfactant adhering freely to the surface of the particles.

The present co-precipitates may optionally be added before and/or during and/or after a buffer/buffer for injectable use, said buffer/buffer capable of making the physiological solutions or at physiological pH.

The present liquid crystals and/or their crystallized dry forms being able to be contained in one and/or more dietetic and/or pharmaceutical forms for oral, parenteral, topical, dermal, transdermal, inhaled, applied to the mucous membranes and to their combinations.

The present liquid crystals and/or their crystallized dry forms being able to be integrated and/or transformed and/or contained in pharmaceutical and/or pharmaceutical and/or dietetic and/or cosmetic forms for oral and/or enteral and/or parenteral and/or injectable and/or cutaneous and/or subcutaneous and/or ocular use and/or applicable to the mucous membranes (buccal, sublingual, lingual, ocular, pulmonary, nasal, vaginal or rectal).

The present liquid crystals and/or their dry crystalline forms (with their included/associated cargoes) being suitable for being incorporated in gastro-resistant capsules and/or envelopes resistant to gastric acid pH and releasing their contents at the intestinal level at neutral or basic pH.

The present liquid crystals and/or their crystallized dry forms being suitable for being incorporated in foods and/or in beverages and/or in candies.

The invention consists of a method making it possible to trap a compound insoluble in a supersaturated solution of betaine and/or in a betaine structure preferably less than 300 nanometers (nm), then to freeze (hold in a fixed state) and/or stabilize this combination by crystallization and/or by crystal growth.

The present liquid crystals and/or their crystallized dry forms, characterized in that they contain and/or comprise and/or encompass and/or are associated with one or more hydrophobic substances having a partition coefficient Log P or Log Kow equal or greater than 1.5, such as greater than 3.

More generally, according to the invention, the present methods for obtaining nucleation and/or germinating and/or co-crystallizations may be extended to all the cargo therapeutic entities.

These methods make it possible to fix (hold in a fixed state) the characteristics present in liquid crystals up to their uses in vivo, thus avoiding the phenomena of coalescence and degradation over time of the liquid forms of the microemulsions. In this important aspect of the invention, the methods allow a greater stability of the galenic and/or therapeutic forms.

The present liquid crystals and/or their crystallized dry forms can be used in the processes of amorphous precipitation: amorphous precipitation product when the drug precipitates in amorphous form in the inert support. The high energy state of the drug in this system generally produces much larger dissolution rates than the corresponding crystalline forms of the drug. The conversion of a drug into an amorphous form leads to its increased dissolution.

The present liquid crystals able to be subjected to one or more crystallizations to obtain a dry product and/or a salt and/or crystals, free and/or substantially free of water and/or solvents.

The present liquid crystals and/or their crystallized dry forms being stable and capable of being resolubilized in one or more solvents, preferably water, and/or one or more physiological liquids.

The present liquid crystals and/or their crystallized dry forms being able to be contained in one or more pharmaceutical forms and/in one or more dietetic forms and/or in one or more cosmetic forms for oral, parenteral, topical, dermal, transdermal, inhaled, pulmonary, applied to the mucous membranes, and to their combinations.

The present liquid crystals and/or their crystallized dry forms being able to be integrated and/or transformed into pharmaceutical and/or pharmaceutical and/or dietetic forms for oral and/or enteral and/or parenteral and/or injectable and/or cutaneous and/or ocular use and/or applicable to the mucous membranes (buccal, sublingual, lingual, pulmonary, nasal, vaginal and/or rectal).

The invention consists of a process for trapping and/or encapsulating and/or transporting a soluble and/or insoluble compound in a supersaturated solution of betaine and/or in a structure having a size preferably of less than 350 nanometers (nm), then optionally of freezing (keeping in a fixed form) and/or of stabilizing this combination by drying and/or dehydration and/or crystallization and/or by crystalline growth.

The size of the structures and/or nanoparticles (comprised) of the preceding paragraph is less than 300 nanometers (nm), preferably less than 250 nm, more preferably less than 200 nm, preferably less than 100 nm, preferably less than 80 nm, more preferably less than 60 nm, advantageously less than 50 nm, preferentially less than 40 nm, even more preferentially less than 20 nm, very preferentially less than 10 nm.

The present liquid crystals and/or their crystallized dry forms, characterized in that they contain and/or comprise and/or encompass one or more hydrophobic substances having a partition coefficient Log P equal or greater than 1.5.

According to the invention, the present liquid crystals and/or their crystallized dry forms and/or their resolubilized dry forms may be characterized by one or more measurements chosen from the group of: solubility of (mg/ml), a size of nanoparticles (DLS), the polydispersity of the nanoparticles, potential Zeta, microcalorimetry, electron microscopy, density, viscosity, surface tension, diffraction, X-ray powder diffraction (XRPD), refraction, reflectivity, photoreactivity, polarization wavelength, conductivity, resistivity, structure, spectroscopy, mass spectroscopy, NMR, HPLC, melting point, cargo, cloud point, cargo, hygroscopicity, volumetric weight, as well as any other measurements known from the state of the art and used to characterize a crystal and/or a liquid crystal and/or a nanoemulsion.

Depending on the hydrophobic substance solubilized each crystal will have a single profile according to the invention.

More generally, the liquid and/or solid crystals of the invention can serve as vectors and/or transports and/or capsules to proteins, metals (radiotherapy), contrast agents, radioelements, fluorophores, DNA and/or RNA sequences, vaccines, viruses and/or bacteria and/or spores and/or fungi, as well as their envelopes and/or fragments; to various entities for therapeutic usesto different entities for dietetic uses, to different entities for chemical purposes, to different entities for electronic uses to different entities for physical uses, to different entities for cosmetic uses, to insecticides, to aromas, to fragrances, to vitamins, to mineral extracts, to plant extracts, to metal extracts, to animal extracts, to human extracts, as well as to the combinations of two or more of the preceding cargoes entities.

According to the invention, the dry and/or substantially dry products of the invention having a particle size of at least 35 mesh, preferably of at least 50 mesh, more preferably of at least 60 mesh, preferably of at least 70 mesh, preferably of at least 80 mesh, more preferably of at least 90 mesh, advantageously of at least 100 mesh, preferentially of at least 125 mesh, even more preferentially of at least 150 mesh, very preferentially of at least 200 mesh.

According to an advantageous aspect of the invention, the finer particle sizes are possible by virtue of the reductions in the amounts of surfactants which allow the synergies of the latter with betaine. The formulations containing more amounts of surfactants prove difficult or even impossible to completely dry, the final products being tacky or viscous due to surfactants.

According to the invention, the particle sizes of the finest dry powders make it possible to optimize the processes of galenical manufacturing and also to have homogeneous powders having defined flows (for example in filling hoppers) and/or physicochemical characteristics necessary or mandatory to respect industry requirements, in particular those of the pharmaceutical industry.

The dry forms of the present liquid crystals are able to be integrated and/or transformed into pharmaceutical and/or pharmaceutical and/or dietetic forms for oral and/or enteral and/or parenteral and/or injectable and/or cutaneous and/or ocular use and/or applicable to the mucous membranes (buccal, sublingual, lingual, pulmonary, nasal, vaginal or rectal).

The liquid crystals and/or their crystallized dry forms of the invention are suitable for being formulated in delayed and/or controlled release and/or sustained release formulations. The releases of one or more cargo entities that can be controlled/prolonged over time and/or controlled/prolonged as regards at the sites of administrations and/or absorptions with, for example, gastro-resistant and/or enterosoluble formulations and/or other similar formulations.

The use of all the excipients (polymers, etc.) known to those skilled in the art for the purposes of the preceding paragraphs are claimed in combinations and/or in combination with the liquid crystals and/or their crystallized dry forms of the invention.

When the above formulations control the release of several cargo entities, the latter may have similar and/or different release profiles, depending on the targets and/or uses and/or the required therapeutic objectives. The present liquid crystals and/or their crystallized dry forms being suitable for being incorporated in food and/or in food supplements and/or in beverages.

The general concept of the present invention is to obtain a liquid crystal and/or a shape and/or a nano-emulsion and/or a crystallogenesis and/or a betaine crystal with remarkable properties and the examples as well as the emulsifiers and co-solutes described here can be extended to all those known by those skilled in the art without being outside the field or outside the concepts described by the present invention.

These methods make it possible to fix the characteristics of the microemulsions and/or the liquid crystals up to their uses in vivo, thus avoiding the phenomena of coalescence and degradation over time of the liquid forms of the microemulsions. In this important aspect of the invention, the methods allow a greater stability of the galenic and/or therapeutic forms.

In an advantageous aspect of the invention, the crystallization processes make it possible to filter and/or attract and/or concentrate and/or trap the entity to be solubilized.

The products and/or methods of the previous one where the dissolution and/or solubilization properties of said products are frozen and/or substantially frozen and/or stabilized over time.

According to embodiments, the present dry and/or substantially dry products may have a stability when they are resolubilized in water over a period of 1 to 36 months; the size of the particles and/or the Zeta potential and/or the polydispersity of the solutions obtained are substantially similar to those of the initial solution with a variation of less 10%, preferably less than 9%, more preferably less than 8%, preferably less than 7%, preferably less than 6%, more preferably less than 5%, advantageously less than 4%, preferentially less than 3%, even more preferentially less than 2%, very preferentially less than 1%.

More generally, according to the invention, the present methods for obtaining nucleation and/or germinating and/or co-crystallizations may be extended to all of the host and/or cargo therapeutic entities.

Combinations/Associations with Cargo Entities

The lack of good aqueous solubility is frequently identified as a major obstacle to the development and clinical use of most (+ of 60%) of the new molecules/therapeutic entities.

Among all the chemical entities recently discovered, about 60% are lipophilic and do not reach the market because of their low water solubility. It is estimated that the future pipeline comprises 90% of poorly soluble compounds.

According to the Biopharmaceuticals Classification System or BCS, a drug is considered to be poorly soluble in water if its maximum therapeutic dose is not soluble in 250 ml or less of aqueous medium in a pH range from 1 to 7.5.

The present liquid crystals have surprisingly been found to be particularly effective in solubilizing hydrophobic substances. They also show a high stability at different pH values ranging from 1 to 8, which corresponds to the acidity/alkalinity range encountered in the living body.

The invention describes compositions for pharmaceutical and/or biochemical and/or biological and/or dietary and/or cosmetic uses comprising a mixture of anhydrous betaine and/or its salts and of a hydrophobic and/or substantially hydrophobic substance having a partition coefficient Log P equal or greater than 1.5, the two ingredients being in a proportion by weight betaine/hydrophobic substance from 200 to 1, preferably from 100 to 1, advantageously from 50 to 1, preferentially from 25 to 1, said composition characterized in that the betaine liquid crystal increases the solubility of the hydrophobic substance in water (for example at room temperature, 20° C.) by a factor of at least 10, preferably by a factor of at least 20, advantageously by a factor of at least 50, preferentially by a factor of at least 100, most preferably by a factor of at least 1,000 compared to the solubility of the hydrophobic substance alone in water (for example at room temperature, 20° C.). The term “drug” is conventional, designating a compound having beneficial prophylactic and/or therapeutic properties when administered to an animal, in particular to humans. Preferably, the drug is a “low water solubility” drug, which means that the drug has a minimum aqueous solubility at a physiologically relevant pH (i.e., pH 1-8) of about 0.1 mg/ml or less.

The invention finds utility when the solubility in water of the drug decreases. Thus, the compositions of the present invention are preferred for low solubility drugs having an aqueous solubility of less than 0.1 mg/ml, advantageously less than 0.05 mg/ml and preferably less than 0.01 mg/ml. The liquid crystals of the invention are particularly useful for serving as transporters and/or vehicles and/or companions and/or vectors and/or containers and/or containers and/or encapsulating and/or protections and/or envelopes with/for different entities having a pharmacological action and/or a therapeutic action and/or a biological action and/or a pharmaceutical action, said entities belonging to different therapeutic classes and/or different dietetics classes and/or different cosmetic classes, as well as their combinations.

According to the invention, the preferred classes of physiologically active molecules and/or entities and/or drugs having a partition coefficient (Log P) equal or greater than 1.5 (advantageously greater than 3), comprise, but are not limited to, anticancer agents, antihypertensive agents, antihypertensive agents for pulmonary hypertension and/or portal hypertension, anxiolytic agents, anticoagulant agents, cardiovascular agents, antiaggregating agents, antidiabetic agents, glucagonanticonvulsants, steroidal agents, diuretic agents, agents antidiuretics, hypoglycemic agents, decongestants, vasodilators, antihistamines, antineoplastic agents, beta-blockers, antipain agents, anti-migraine agents, anti-inflammatory agents, non-steroidal anti-inflammatory agents, corticoids, corticosteroids, antipyretics, antipsychotics, antidepressants, sleeping pills, calming agents, anorexigens, opioid analgesics, psychotropic agents, cognition enhancers anti-spasmodic agents, antiepileptic agents, cholesterol-reducing agents, statins, anti-atherosclerosis agents, anti-obesity agents, anti-metabolic syndrome agents, agents regulating metabolism, anti-diarrhea agents, insulin sensitizing agents and/or potentiating the effects of insulin, anti-osteoporosis agents, anti-asthma agents, hepatoprotective agents, anti-autoimmune disease agents, anti-cystic fibrosis agents, anti-cystic fibrosis agents, anti-hereditary diseases, anti-neurodegenerative agents, anti-acne agents, immunosuppressive and/or immunomodulatory agents, anti-transplant rejection agents, enzymatic agents, ciclosporins, gene therapy agents, anti-sexual impotence agents, erectile dysfunction agents, growth hormones, contraception agents, anti-thyroid agents, anti-dripping agentsanti-anginal agents, acetylcholinesterase inhibitors, anthelmintics, anti-prostate hypertrophy agents, anti-muscarinic agents, antiarrhythmic agents, antimalarial agents, neuroleptic agents, cardiac inotropic agents, gastrointestinal agents, histamine antagonists, muscle relaxants, stimulants such as amphetamine, dexamphetamine, dexfenfluramine, fenfluramine and mazindol, antibacterial agentsantifungal agents, antiviral agents, hypnotic agents, narcotic agents, opiates, anesthetics, antiparkinsonian agents, anti-Alzheimer's disease agents, antibiotics, hormones, sex hormones (testosterone, progesterone, etc.), antitussive agents, protease inhibitors, glycogen phosphorylase inhibitors, cholesteryl ester transfer protein inhibitors, antiprotozoal agents, anti-malaria agents, anti-infective agents, antiparasitic agents, anti-baldness agents, anti-alopecia agents, antidotes, alkaloids, contrast agents, imaging and/or medical acquisition agents, magnetic resonance agentsmagnetic and/or mineral agents, magnetic and/or mineral agents, metal and/or mineral and/or synthetic agents for magnetic and/or wave and/or heat treatments, vaccines, enzymes, peptides, RNA sequences, DNA sequences, antibodies, vitamins, polyphenols, anthocyanins, nutraceutical and/or dietetic agents of mineral and/or animal and/or plant and/or synthetic origin, plant substancesplant extracts, nutraceutical agents such as stilbenes, carotene, flavonoids, DHEA, ginseng, polyphenols and similar substances, mineral substances, metals, antioxidants, coenzyme Q10, natural and/or synthetic extracts of turmeric such as curcumin, Curcuminoids the phenolic compounds of turmeric, desmethoxycurcumin, bisdemethoxycurcumin, natural cannabis extracts, synthetic cannabis extracts, natural and/or synthetic extracts of coca such as cocaine, natural and/or synthetic extracts of Taxol, natural and/or synthetic extracts of Cannabis Sativa and/or Cannabis Indica and/or Cannabis Ruderalis and/or hybrids thereof and/or crosses thereof, fragrances, essential oils, flavors, body hygiene agents, phytosanitary agents, insecticides, cosmetic agents; as well as the combinations of two or more treatments and/or molecules and/or entities and/or agents of the preceding list.

Accordingly, methods of treatments using one or more agent from the preceding list/paragraph in combination with the liquid crystals of the invention, are claimed. Such methods of treatments being particularly suitable in related conditions and/or diseases for increasing the safety and/or the efficacy and/or the bioavailability and/or the compliance for a subject in need.

The invention is advantageously useful for solubilizing and/or increasing the bioavailability of the treatments and/or molecules and/or entities and/or agents, as well as their salts and/or isomers and/or esters thereof, and/or ethers and/or derivatives thereof, of the preceding paragraph belonging to Classes II and IV of BCS.

According to another aspect of the invention, entities belonging to the Class III of BCS (soluble but having a low membrane permeability and/or membrane passage) may also benefit from the advantages of the invention in particular with regard to the protection of these entities with respect to enzymatic degradation and/or metabolic degradation and/or their better intestinal absorption for the purpose of better bioavailability and/or prolonged and/or modulated half-life in the body.

According to the invention, the present liquid crystals make it possible to increase and/or optimize the membrane permeability and/or the membrane passage of the cargo entities.

Preferably, the invention relates to molecules and/or entities having an oral bioavailability of less than 70%, 60%, 50%, 40%, 30%, 20%, 15%, 10%, 5%, etc. According to the invention, the present liquid crystals can transport and/or be associated with one or more cargo entities selected from the groups consisting of (i) acetylcholinesterase inhibitors selected from the group comprising donepezil, tacrine and pyridostigmine; (ii) non-steroidal anti-inflammatory agents and anti-inflammatory agents (NSAIA) selected from the groups: aloxiprin, auranofin, azapropazone, ibuprofen, fenoprofen, benorylate, capsaicin, diclofenac, naproxen, naproxone, flunomide, meclofenamic acid, mefenamic acid, nabutta, ivermectin, mebendazole, oxamniquine, oxfendazole, oxantel embonate, praziquantel, pyrantel embonate and thiabendazole; (iv) antiacne agents selected from the group consisting of isotretinoin and tretinoin; (v) anti-anginal agents selected from the group comprising amyl nitrate, glyceryl trinitrate (nitroglycerin), isosorbide dinitrate, isosorbide mononitrate, pentaerythritol tetranitrate and ubidecarenone (coenzyme Q10); (vi) antiarrhythmic agents selected from the group comprising amiodarone HCl, digoxin, disopyramideand quinidine sulfate; (vii) anti-asthmatic agents selected from the group consisting of zileuton, zafirlukast, terbutaline sulfate, montelukast and albuterol; (viii) antibacterial agents, including antibiotics, selected from the group consisting of alatroflox, azithromycin, aztreonum, baclofen, benzathine penicillin, clofazimine, cefixime, ofloxacin, ethoxazole, sulphapyridine, tetracycline, trimethoprim, trovafloxacin and vancomycin; (ix) benign prostatic hypertrophy (HBP) agents selected from the group consisting of alfuzosin, doxazosin, phenoxybenzamine, prazosin, terrazosin and tamulosin; (x) anticancer and immunosuppressive agents selected from the group consisting of abarelix, aldesleukin, alemtuzumab, alitretinoid, altramine, amifostine, aminoglutethimide, amsacrine, anastrozole, arsenic oxide, asparaginase, amalguthimide, amidoglutethimideazurantine, bicalutamide, bisantrene, bleomycin, bortezomib, busulfan, calusterone, camptothecin, capecitabine, carboplatin, carmustine, celecoxib, cetuximab, chlorambucil, cisplatin, cladribine, cladarin, clofarabine, cyclofosphamine, cyclophosphamine, cyclosporine, cytarabine, dacarbazine, dactinomycin, darbepoetin alpha, daunorubicin, denileukin, dexrazoxane, docetaxel, doxorubicin (neutral), doxorubicin HCl, dromostanolone propionate, ellipticine, enlimomab, estramustine, epirubicin, epoetin alfa, erlotinib, estramustine, etoposide, exemestane, filgrastim, floxacin, fludarabine, fulvestrant, gefitinib, gemcitabine, gemtuzumab, goserelin acetate, hister acetate, hydroxyurea, ibritumomab, idarubicin, ifosfamide, imatinib mesylate, interferon alfa-2 a, interferon alfa-2 b, interferon alfa-2 b, irorefercan, lenalidomide, letrozole, leucovorin, leuprolide acetate, levamisole, lomustinemegestrol acetate, melphalan, mercaptopurine, mesna, methotrexate, methoxsalen, mitomycin C, mitotane, mitoxantrone, mofetimycophenolate, nandrolone, nelarabine, nilutamide, nofetumomab, oprelvekin, oxaliplatin, taxol, taxanes, paclitaxel, palifermine, pamidronate, pegadenmase, pegaspargase, pegfilgrastim, pemetrexed disodium, pentostatin, pipobroman, plicamycin, sodium porfimer, procarbazine, quinacrine, rasburicase, rituximab, sargramostim, sirolimus, sorafenib, streptozocine, sunitinib maleate, tacrolimus, tamoxifen, tamoxifen citrate, temozolomide, teniposide, testolactone, thioguanine, thiotepa, topotecan, toremifene, tositumomab, trastuzumab, tretinoin, ATRA, uracil mustard, valrubicin, vinblastine, vincristine, vinorelbine, zoledronate, and zoledronic acid; (xi) the anticoagulants selected from the group consisting of aspirin, cilostazol, clopidogrel, dicoumarol, dipyridamole, nicoumalone, oprelvekin, phenindione, ticlopidine and tirofiban; (xii) antidepressants selected from the group consisting of amoxapine, bupropion, citalopram, clomipramine, fluoxetine HCl, maprotiline hydrochloride, mianserin HCl, nortriptyline HCl, paroxetine HCl, sertraline HCl, trazodone hydrochloride, trimipramine maleateand venlafaxine HCl; (xiii) antidiabetics selected from the group consisting of insulin, acetohexamide, chlorpropamide, glibenclamide, gliclazide, glipizide, glimepiride, glyburide, miglitol, pioglitazone, repaglinide, rosiglitazone, tolazamide, tolbutamide and troglitazone; (xiv) antiepileptics selected from the group consisting of beclamide, carbamazepine, clonazepam, thotoin, felbamate, fosphenyloin sodium, lamotrigine, method, methsuximide, methylphenobarbitone, oxcarbazepine, paramethadione, phenacenmide, phenobarbital, phenyloin, phensuximide, primidone, sulthiame, tiagabineHCl, topiramate, valproic acid, and vigabatrin; (xv) antifungal agents selected from the group consisting of amphotericin, butenafine HCl, butoconazole nitrate, clotrimazole, econazole nitrate, fluconazole, flucytosine, griseofulvin, itraconazole, ketoconazole, miconazole, natamycin, nystatin, sulconazole nitrateoxiconazole, terbinafine hydrochloride, terconazole, tioconazole and undecenoic acid; (xvi) anti-dripping agents selected from the group consisting of a llopurinol, the problem and sulfinpyrazone; (xvii) antihypertensive agents selected from the group consisting of amlodipine, bennidipine, benezepril, candesartan, captopril, darodipine, dilitazem HCl, diazoxide, doxazosin HCl, enalapril, eposartan, losartan mesylate, felodipine, fenoldopamfosopril, guanabenz acetate, irbesartan, isradipine, lisinopril, minoxidil, nicardipine HCl, nifedipine, nimodipine, nisoldipine, phenoxybenzamine HCl, HCl prazosin, quinapril, serpin, terrazosine HCl, telmisartan and valsartan; (xviii) antimalarial agents selected from the group consisting of amodiaquine, chloroquine, chlorproguanil hydrochloride, halofantrin HCl, mefloquine hydrochloride, proguanil HCl, pyrimethamine, quinine and quinine sulfate; (xix) antimigraine agents selected from the group consisting of dihydroergotamine mesylate, ergotamine tartrate, frovatriptan, methysergide maleate, naratriptan HCl, pizotifen maleate, rizatriptan benzoate, sumatriptan succinate, and zolmitriptan; (xx) the anti-muscarinic agents selected from the group consisting of atropine, benzhexol HCl, biperiden, ethpropazine hydrochloride, hyoscyamine, mepenzolate bromide, oxyphencyclimine HCl and antiparkinsonian agents tropicamide (xxi) selected from the group consisting of bromocriptine mesylate, maleate lysuride, pramipexole, ropinirole HCl and tolcapone; (xxii) antiprotozoal agents selected from the group consisting of atovaquone, benznidazole, clioquinol, decoquinate, diiodohydroxyquinoline, diloxanidemometasone, dinitolmidefurazolidone, metronidazole, nimorazole, nitrofurazone, ornidazole and tinidazole; (xxiii) the anti-thyroid agents selected from the group consisting of carbimazole and propylthiouracil; (xxiv) an antitussive agent such as benzonatate; (xxv) antiviral agents selected from the group consisting of abacavir, amprenavir, delavirdine, efavirenz, indinavir, lamivudine, nelfinavir, nevirapine, ritonavir, saquinavir, stavudine and; (xxvi) anxiolytics, sedatives, hypnotics and neuroleptics selected from the group consisting of alprazolam, amylobarbitone, barbitone, bentazepam, bromazepam, bromperidol, brotizolam, bubbitone, carbromal, chlordiazepoxide, chlormethiazole, chlorpromazine, chlorprothixen, clonazepam, clobazam, clotiazepam, clozapine, diazepam, droperidol, ethinamate, flunanisone, flunitrazepam, triflupromazine, flupenthixol decanoatefluphenthixol decanoate, flurazepam, gabapentin, haloperidol, lorazepam, lormetazepam, medazepam, meprobability, mesoridazine, methaqualone, methylphenidate, midazolam, molindone, nitrazepam, olanzapine, oxazepam, pentobarbitone, perphenazinepimozide, prochlorperazine, propofol, pseudoephedrine, quetiapine, risperidone, sertindole, sulpiride, temazepam, thioridazine, triazolam, zolpidem, zopiclone and; (xxvii) b-blockers selected from the group consisting of acebutolol, alprenolol, atenolol, betalol, metoprolol, nadolol, oxprenolol, pindolol and propranolol; (xxviii) cardiac inotropic agents selected from the group consisting of anrinone, digitoxin, digoxin, enoximone, lanatoside C and medigoxin; (xxix) corticosteroids selected from the group consisting of beclomethasone, betamethasone, budesonide, cortisone acetate, deoxymethasonedexamethasone, fludrocortisone acetate, flunisolide, fluocortolone, fluticasone propionate, hydrocortisone, methylprednisolone, prednisolone, prednisone and triamcinolone; (xxx) diuretics selected from the group consisting of acetazolamide, amiloride, bendroflumethiazide, bumetanide, chlorothiazide, chlorthalidone, ethacrynic acid, furosemide, metolazone, spironolactone and triamterene; (xxxi) the gastrointestinal agents selected from the group consisting of bisacodyl, cimetidine, cisapride, diphenoxylate hydrochloride, domperidone, famotidine, lanosprazole, loperamide, mesalazine, nizatidine, omeprazole, ondansetron hydrochloride, pantoprazole, rabeprazole sodium, ranitidine HCl and sulfasalazine; (xxxii) histamine H2 hsub antagonists 1-receptors selected from the group consisting of acrivastine, astaxanthin, chlorpheniramine, cinnarizine, cetrizine, clemastine fumarate, cyclizine, cyproheptadine hydrochloride, dexchlorpheniramine, dimenhydrinate, fexofenadine, flunarizine HCl, loratadine, HCl meclizine, oxatomide and terfenadine; (xxxiii) keratolytic agents selected from the group consisting of acetretin, calciprotriene, calcifediol, calcitriol, cholecalciferol, ergocalciferol tretinate, retinoids, Targretin, and tazarotene; (xxxiv) lipid regulating agents/hypolipidemic agents selected from the group consisting of statins, atorvastatin, bezafibrate, cerivastatin, ciprofibrate, clofibrate, fenofibrate, fluvastatin, gemfibrozil, hesperetin, lovastatin, pravastatin, probucol and simvastatin; (xxxv) myorelaxants selected from the group consisting of cyclobenzaprine, dantrolene sodium and tizanidineHCl; (xxxvi) opioid analgesics selected from the group consisting of codeine, dextrorpropoxyphene, diamorphine, dihydrocodeine, fentanyl, meptazinol, methadone, morphine, nalbuphine and pentazocine; (xxxvii) hormones and sex hormones selected from the group consisting of clomifen citrate, cortisone acetate, danazol, dehydroepiandrosterone, ethynyl estradiol, finasteride, fludrocortisone, fluoxymesterone, medroxyprogesterone acetate, megestrol acetate, mestranol, methyltestosterone, mifepristone, norethisterone, norgestrel, estradiol, conjugated estrogens, progesterone, rimexolone, stanozolol, stilbestrol, testosterone and tibolone; (xxxviii) stimulants selected from the group consisting of amphetamine, dexamphetamine, dexfenfluramine, fenfluramine and mazindol; (xxxix) nutraceuticals selected from the group consisting of calcitriol, carotene, chrysin, dihydrotachysterol, flavonoids, hesperetin, jasmonates, lipoic acid, lutein, lycopene, essential fatty acids, non-essential fatty acids, naringenin, phytonadiol, quercetin, vitamins, including vitamin A, vitamin B2, vitamin D and its derivatives, vitamin E, and vitamin K, coenzyme Q10 (ubiquinone), polyphenols, flavonoids, catechins, epicatechins, epicatechins gallate, quercetin, resveratrol, lycopene, lutein, polyunsaturated fatty acids, b-carotene or vitamin a, alpha-tocopherol or vitamin E, Vitamin d, isoflavones, leuprolide acetate, buprenorphine, peptides, proteins, DNA and/or RNA sequences, plant extracts, minerals, therapeutic agents used in immunology, neurology-psychiatric, ophthalmology, HIV protease inhibitors, menopause, rheumatism, sleep disorders, erectile dysfunction, osteoporosis, thyroid disorders, vaccination, venous insufficiency (veinotonic), stress, osteoarthritis, Errata and/or Androgenic Alopecia, Rhumatisms, Tabagismus; (xxxxi) the nutraceuticals such as calcitriol, carotene, chrysin, dihydrotachysterol, flavonoids, hesperetin, jasmonates, lipoic acid, lutein, lycopene, essential fatty acids, non-essential fatty acids, naringenin, phytonadiol, quercetin, vitamins, including vitamin A, vitamin B2, vitamin D and its derivatives, vitamin E, and vitamin K, coenzyme Q10 (ubiquinone), plant extracts, metals and minerals; as well as the combination and/or combinations of the ingredients listed above.

Accordingly, methods of treatments using one or more agent from the preceding list/paragraph in combination with the liquid crystals of the invention, are claimed. Such methods of treatments being particularly suitable in related conditions and/or maladies for increasing the safety and/or the efficacy and/or the bioavailability and/or the compliance for a subject in need.

This list is only by way of example and in no case limits the scope of the invention to the other entities/molecules of the same class having a Log P greater than or equal to 1.5.

Since this list is not exclusive, any similar therapeutic entity may be integrated therein so that said entity encounters, after administration in the host, problems of solubility and/or bioavailability and/or efficacy and/or toxicity and/or half-life and/or degradation which would be linked to its structure and/or to its lipophilicity. According to the invention, the cargoes nutraceutical agents may be chosen for example from the groups comprising calcitriol, carotene, chrysin, dihydrotachysterol, flavonoids, hesperidin, jasmonates, lipoic acid, lutein, lycopene, essential fatty acids, non-essential fatty acids, naringenin, phytonadiol, quercetin, vitamins, including vitamin A, vitamin B2, vitamin D and its derivatives, vitamin E, and vitamin K, coenzyme Q10 (ubiquinone), as well as the combinations of two or more treatments and/or molecules and/or entities and/or agents of the preceding vitamin list, as well as in combinations with the other entities and/or agents of different classes (anticancer, anti-inflammatory, etc.) described and listed in the present document.

This list is only by way of example and in no case limits the scope of the invention to the other entities/molecules of the same class having a Log P equal or greater than to 2.

The metals and/or minerals such, iron, gold, silver, zinc, platinum, copper, hafnium oxide, barium, may be encapsulated by the liquid crystals of the invention.

The anticancer and/or immunosuppressive agents may, for example, be chosen from the groups comprising abarelix, aldesleukin, alemtuzumab, alitretinoin, altramine, amifostine, aminoglutethimide, amsacrine, anastrozole, arsenic oxide, asparaginase, amalguthimide, amidoglutethimide, azurantine and bicalutamide health agent, bisantrene, bleomycin, bortezomib, busulfan, calusterone, camptothecin, capecitabine, carboplatin, carmustine, celecoxib, cetuximab, chlorambucil, cisplatin, cladribine, cladarin, clofarabine, cyclofosphamine, cyclophosphamine, cyclosporine, cytarabine, dacarbazine, dactinomycin, darbepoetin alpha, daunorubicin, denileukin, dexrazoxane, docetaxel, doxorubicin (neutral), doxorubicin HCl, dromostanolone propionate, ellipticine, enlimomab, estramustine, epirubicin, epoetin alfa, erlotinib, estramustine, etoposide, exemestane, filgrastim, flodarabine, fulvestrant, gefitinib, gemcitabine, gemtuzumab, goserelin acetate, hister acetate, hydroxyurea, ibritumomab, idarubicin, ifosfamide, imatinib mesylate, interferon alfa-2 a, interferon alfa-2 b, interferon alfa-2 b, irorefercan, lenalidomide, letrozole, leucovorin, leuprolide acetate, levamisole, lomustine, megestrol acetate, melphalan, mercaptopurine, mesna, methotrexate, methoxsalen, mitomycin C, mitotane, mitoxantrone, mofetimycophenolate, nandrolone, nelarabine, nilutamide, nofetumomab, oprelvekin, oxaliplatin, paclitaxel, palifermine, pamidronate, pegadase, pegaspargase, pegfilgrastim, pemetrexed disodium, pentostatin, pipobroman, plicamycin, sodium porfimer, procarbazine, quinacrine, rasburicase, rituximab, sargramostim, sirolimus, sorafenib, streptozocine, sunitinib maleate, tacrolimus, tamoxifen, tamoxifen citratetemozolomide, teniposide, testolactone, thioguanine, thiotepa, topotecan, toremifene, tositumomab, trastuzumab, tretinoin, ATRA, uracil mustard, valrubicin, vinblastine, vincristine, vinorelbine, zoledronate, zoledronic acid, as well as the combinations of two or more treatments and/or molecules and/or entities and/or agents of the preceding list. This list is only by way of example and in no case limits the scope of the invention to the other entities/molecules of the same class having a Log P greater than or equal to 1.5.

Accordingly, methods of treatments using one or more agents from the preceding list/paragraph in combination with the liquid crystals of the invention, are claimed. Such methods of treatments being particularly suitable in related conditions and/or maladies for increasing the safety and/or the efficacy and/or the bioavailability and/or the compliance for a subject in need.

The advantage of the invention is to be able to freeze (hold in a fixed state) and/or fix the properties of the solutions obtained by crystallizing them, preventing them from degrading over time following the known phenomena of decomposition, oxidation, deliquescence, coalescence, aggregation, flocculation and “Otswald ripening” that the liquid forms do not lack any sustained periods.

Very advantageously, this process by fixing the solutions obtained in dry and/or crystalline forms, makes it possible to retain and/or fix and/or stabilize and/or prolong their pharmacological and/or chemical and/or dynamic and/or biological properties over time.

Very advantageously, these pharmacological and/or chemical and/or thermodynamic properties are directly restored when the dry and/or crystalline forms are resolubilized.

According to the invention, the polymers and/or additives and/or excipients generally known in the art of the formulation and/or in the art of the galenic may be optionally added.

In general, excipients such as fillers, disintegrating agents, pigments, binders, lubricants, glidants, flavoring agents, etc. can be used for the usual purposes and the typical quantities of the art, without harming the properties of the compositions.

These excipients may be used before and/or during and/or after the formation of the composition in order to formulate it in tablets, capsules, suppositories, suspensions, powders for suspension, creams, transdermal patches, patches, sprays, injectable products, sublingual products, drops, dyes, gels, ointments, deposits, etc.

The compositions of the present invention may be formulated in various forms as delivered as a suspension of particles in a liquid vehicle. These suspensions may be formulated in liquid or paste form at the time of manufacture, or in dry powder form with a liquid, generally water, added at a later time, but before oral administration.

These powders constituted in suspension are sachets or formulations of oral powder for constituting (OPC). Such dosage forms may be formulated and reconstituted. The simplest approach consists in formulating the dosage form in the form of a reconstituted dry powder by simple addition of water and stirring.

Alternatively, the dosage form may be formulated as a liquid and dry powder which are combined and agitated to form the drinkable suspension. In yet another embodiment, the dosage form may be formulated as two powders that are reconstituted by first adding water to a powder to form a solution to which the second powder is combined with agitation to form the suspension.

The dosage forms may be protected by a film and/or a barrier and/or a shell having an adequate MVTR. A high MVTR for protecting these forms against moisture.

In general, it is preferable for the drug dispersion to be formulated for long-term storage in the dry state because this promotes the chemical and physical stability of the drug.

Pharmaceutical and/or Therapeutic Uses

The liquid crystals of the invention are particularly useful for serving as transporters and/or companies and/or vectors and/or containers and/or containers and/or encapsulants and/or protections and/or envelopes to various cargo entities having a pharmacological action and/or a therapeutic action and/or a biological action and/or a pharmaceutical action.

According to this advantageous aspect, the liquid crystals of the invention can optimize the action of the cargo entities, both at the levels of their solubilities and/or their bioavailability and/or their stability, and/or their half-lives and/or their safety and/or the reduction of their undesirable effects and/or the increase in their efficiencies and/or their acceptability in living beings.

According to the invention, these cargoes entities and/or entities may be chosen alone and/or in combinations in the groups comprising: (i) acetylcholinesterase inhibitors such as donepezil, tacrine and pyridostigmine; (ii) analgesics (iii) non-steroidal anti-inflammatory agents (NSAIDs) such as aloxiprin, auranofin, azapropazone, benorylate, capsaicin, diclofenac, aceclofenac, indomethacin, ketoprofen, flunomide, meclofenamic acid, acid mefenamic, nabumet, ivermectin, mebendazole, oxamniquine, oxfendazole, oxantel embonate, praziquantel, pyrantel embonate and thiabendazole; (iv) antiacne agents such as isotretinoin and tretinoin; (v) anti-anginal agents such as amyl nitrate, glyceryl trinitrate (nitroglycerin), isosorbide dinitrate, isosorbide mononitrate, pentaerythritol tetranitrate and lubidecarenone (coenzyme Q10); (vi) antiarrhythmic agents such as amiodarone HCl, digoxin, disopyramide, flecainide acetate and quinidine sulfate; (vii) anti-asthmatic agents such as zileuton, zafirlukast, terbutaline sulfate, montelukast and albuterol; (viii) antibacterial agents, including antibiotics, such as alatroflox, azithromycin, aztreonum, baclofen, benzathine penicillin, clofazimine, cefixime, ethical-oxazole, sulphapyridine, tetracycline, trimethoprim, trovafloxacin and vancomycin; (ix) benign prostatic hypertrophy (HBP) agents such as alfuzosin, doxazosin, phenoxybenzamine, prazosin, terrazosin and tamulosin; (x) anticancer and immunosuppressive agents such as abarelix, aldesleukin, alemtuzumab, alitretinoin, altramine, amifostine, aminoglutethimide, amsacrine, anastrozole, arsenic oxide, asparaginase, amalguthimide, amidoglutethimide, azurantine, bicalutamide, bisantrene, bleomycinbortezomib, busulfan, calusterone, camptothecin, capecitabine, carboplatin, carmustine, celecoxib, cetuximab, chlorambucil, cisplatin, cladribine, cladarin, clofarabine, cyclofosphamine, cyclophosphamine, cyclosporine, cytarabine, dacarbazine, dactinomycin, darbepoetin alpha, daunorubicin, denileukin, dexrazoxane, docetaxel, doxorubicin (neutral), doxorubicin HCl, dromostanolone propionate, ellipticine, enlimomab, estramustine, epirubicinepoetin alfa, erlotinib, estramustine, etoposide, exemestane, filgrastim, floxacin, fludarabine, fulvestrant, gefitinib, gemcitabine, gemtuzumab, goserelin acetate, hister acetate, hydroxyurea, ibritumomab, idarubicin, ifosfamide, imatinib mesylate, interferon alfa-2 a, interferon alfa-2 b, interferon alfa-2 b, irorefercan, lenalidomide, letrozole, leucovorin, leuprolide acetate, levamisole, lomustine, megestrol acetate, melphalan, mercaptopurine, mesna, methotrexate, methoxsalen, mitomycin C, mitotane, mitoxantrone, mofetimycophenolate, nandrolone, nelarabine, nilutamide, nofetumomab, oprelvekin, oxaliplatin, paclitaxel, palifermine, pamidronate, pegadenmase, pegaspargase, pegfilgrastim, pemetrexed disodium, pentostatin, pipobroman, plicamycin, sodium porfimer, procarbazine, quinacrine, rasburicase, rituximab, sargramostim, sirolimus, sorafenib, streptozocine, sunitinib maleate, tacrolimus, tamoxifen citrate temozolomide, teniposide, testolactone, thioguanine, thiotepa, topotecan, toremifene, tositumomab, trastuzumab, tretinoin, ATRA, uracil mustard, valrubicin, vinblastine, vincristine, vinorelbine, zoledronate, and zoledronic acid; (xi) the anticoagulants and/or antiaggregating agents chosen such as cilostazol, aspirin, clopidogrel, dicoumarol, simtron, anti-vitamins K, warfarin, fluindione, acetocoumarol, dipyridamole, nicoumalone, oprelvekin, phenindione, ticlopidine and tirofiban; (xii) antidepressants such as amoxapine, bupropion, citalopram, clomipramine, fluoxetine HCl, maprotiline hydrochloride, mianserin HCl, nortriptyline HCl, paroxetine HCl, sertraline HCl, trazodone hydrochloride, trimipramine maleate, and venlafaxine HCl; (xiii) selected antidiabetic agents such as acetohexamide, metformin, chlorpropamide, glibenclamide, gliclazide, glipizide glimepiride, glyburide, miglitol, pioglitazone, repaglinide, rosiglitazone, tolazamide, tolbutamide and troglitazone; (xiv) antiepileptics such as beclamide, carbamazepine, clonazepam, thotoin, felbamate, fosphenyloin sodium, lamotrigine, method, methsuximide, methylphenobarbitone, oxcarbazepine, paramethadione, phenacabine HCl, topiramate, phenyloin, phensuximide, primidone, sulthiame, tiagabine HCl, topiramate, valproic acid, and vigabatrin; (xv) antifungal agents such as amphotericin, butenafine HCl, butoconazole nitrate, clotrimazole, econazole nitrate, fluconazole, flucytosine, griseofulvin, itraconazole, ketoconazole, miconazole, natamycin, nystatin, sulconazole nitrate, oxiconazole, terbinafine hydrochloride, terconazole, tioconazole and undecenoic acid; (xvi) anti-dripping agents such as allopurinol, the problem and sulfinpyrazone; (xvii) antihypertensive agents such as amlodipine, benidipine, benezepril, candesartan, captopril, darodipine, dilitazem HCl, diazoxide, doxazosin HCl, enalapril, eposartan, losartan mesylate, felodipine, fenoldopam, fosopril, guanabenz acetate, irbesartan, isradipine, lisinopril, minoxidil, nicardipine HCl, nifedipine, nimodipine, nisoldipine, phenoxybenzamine HCl, prazosin HCl, quinapril, serpin, terazosin HCl, telmisartan and valsartan; (xviii) antimalarial agents such as amodiaquine, chloroquinechlorproguanil hydrochloride, halofantrine HCl, mefloquine hydrochloride, proguanil HCl, pyrimethamine and quinine sulfate; (xix) antimigraine agents such as dihydroergotamine mesylate, ergotamine tartrate, frovatriptan, methysergide maleate, naratriptan HCl, pizotifen maleate, rizatriptan benzoate, sumatriptan succinate, and zolmitriptan; (xx) antimuscarinic agents such as atropine, benzhexol HCl, biperiden, hydrochloride of ethpropazine, hyoscyamine, mepenzolate bromide, oxyphencyclimine HCl and antiparkinsonian agents tropicamide (xxi) the antiparkinsonian agents such as bromocriptine mesylate, Biperiden, Scopolamine, Trihexyphenidyl, Tropaterpine, Apomorphine, bromocriptine, Lisuride, Pergolide, Piribedil, Pramipexole, Ropinirole, Rotigotine, Safinamide, Amantadine, Levodopa+dopadencarboxylase inhibitor, Levodopa+dopadencarboxylase inhibitor+1-COMT, Entacaponetolcapone, rasagiline, selegiline, lysuride maleate, pramipexole, ropinirole HCl and tolcapone; (xxii) antiprotozoal agents such as atovaquone, benznidazole, clioquinol, decoquinate, diiodohydroxyquinoline, diloxanidemometasone, dinitolmide, furazolidone, metronidazole, nimorazole, nitrofurazone, ornidazole and tinidazole; (xxiii) anti-thyroid agents such as carbimazole and propylthiouracil; (xxiv) antitussive agents such as benzonatate; (xxv) antiviral agents such as abacavir, amprenavir, delavirdine, efavirenz, indinavir, lamivudine, nelfinavir, nevirapine, ritonavir, saquinavir, stavudine; (xxvi) anxiolytics, sedatives, hypnotics and neuroleptics such as alprazolam, amylobarbitone, barbitone, bentazepam, bromazepam, bromperidol, brotizolam, bubbitone, carbromal, chlordiazepoxide, chlormethiazole, chlorpromazine, chlorprothixen, clonazepam, clobazam, clotiazepam, clozapine, diazepam, droperidol, ethinamate, flunanisone, flunitrazepam, triflupromazine, flupenthixol decanoate, fluphenthixol decanoate, flurazepam, gabapentin, haloperidol, lorazepam, lormetazepam, medazepam, mefenamate, mesoridazine, methaqualone, methylphenidate, midazolam, molindone, nitrazepam, olanzapine, oxazepam, pentobarbitone, perphenazinepimozide, prochlorperazine, propofol, pseudoephedrine, quetiapine, risperidone, sertindole, sulpiride, temazepam, thioridazine, triazolam, zolpidem, zopiclone; (xxvii) b-blocking agents such as acebutolol, alprenolol, atenolol, labpetalol, metoprolol, nadolol, oxprenolol, pindolol and propranolol; (xxviii) cardiac inotropic agents such as anrinone, digitoxin, digoxin, enoximone, lanatoside C and medigoxin; (xxix) corticosteroids such as betamethasone, betamethasone, budesonide, cortisone acetate, dexamethasone, dexamethasonefluocortisone acetate, flunisolide, fluocortolone, fluticasone propionate, hydrocortisone, methylprednisolone, prednisolone, prednisone and triamcinolone; (xxx) diuretics such as acetazolamide, amiloride, bendroflumethiazide, bumetanide, chlorothiazide, chlorthalidone, ethacrynic acid, furosemide, metolazone, spironolactone and triamterene; (xxxi) gastrointestinal agents such as bisacodyl, cimetidine, cisapride, diphenoxylate hydrochloride, domperidone, famotidine, lanosprazole, loperamide, mesalazine, nizatidine, omeprazole, ondansetron hydrochloride, pantoprazole, rabeprazole sodium, ranitidine HCl and sulfasalazine; (xxxii) histamine H2 hsub antagonistsl-receptors such as acrivastine, astaxanthin, chlorpheniramine, cinnarizine, cetrizine, clemastine fumarate, cyclizine, cyproheptadine hydrochloride, dexchlorpheniramine, dimenhydrinate, fexofenadine, flunarizine HCl, loratadine, HCl meclizine, oxatomide and terfenadine; (xxxiii) keratolytic agents such as acetretin, calciprotriene, calcifediol, calcitriol, cholecalciferol, ergocalciferol, etretinate, retinoids, targretin, and tazarotene; (xxxiv) lipid regulating agents/hypolipidemic agents such as statins, atorvastatin, bezafibrate, cerivastatin, ciprofibrate, clofibrate, fenofibrate, fluvastatin, gemfibrozil, hesperetin, lovastatin, pravastatin, probucol and simvastatin; (xxxv) myorelaxants such as cyclobenzaprine, dantrolene sodium and tizanidine HCl; (xxxvi) opioid analgesics such as codeine, dextrorpropoxyphene, diamorphine, dihydrocodeine, fentanyl, meptazinol, methadone, cocaine, lidocaine, morphine, nalbuphine and pentazocine; (xxxvii) sex hormones such as clomifen citrate, cortisone acetate, danazol, dehydroepiandrosterone, ethynyl estradiol, finasteride, fludrocortisone fluoxymesterone, medroxyprogesterone acetate, megestrol acetate, mestranol, methyltestosterone, mifepristone, norethisterone, norgestrel, estradiol, conjugated estrogens, progesterone, rimexolone, stanozolol, stilbestrol, testosterone and tibolone; (xxxviii) stimulants such as amphetamine, dexamphetamine, dexfenfluramine, fenfluramine and mazindol; (xxxix) cholinesterase inhibitors in Alzheimer's disease such as donepezil, galantamine, rivastigmine; (xxxx) the therapeutic agents used in Immunology, Neurology-psychiatric, Ophthalmology, Menopause, Rheumatism, Sleep Disorders, Erectile Disorders, Osteoporosis, Fibromyalgia Disorders of the Thyroid, Vaccination, Venous Insufficiency (Veinotonic), Stress, Osteoarthritis, Alopecia Errata and/or Androgenic, Rhumatisms, Tabagismus; (xxxxi) the nutraceuticals such as calcitriol, carotene, chrysin, dihydrotachysterol, flavonoidshesperetin, jasmonates, lipoic acid, lutein, lycopene, essential fatty acids, non-essential fatty acids, naringenin, phytonadiol, quercetin, vitamins, including vitamin A, vitamin B2, vitamin D and derivatives thereof, vitamin E, and vitamin K, coenzyme Q10 (ubiquinone), plant extracts, metals and minerals.

Accordingly, methods of treatments using one or more agent from the preceding list/paragraph in combination with the liquid crystals of the invention, are claimed. Such methods of treatments being particularly suitable in related conditions and/or diseases for increasing the safety and/or the efficacy and/or the bioavailability and/or the compliance for a subject in need.

The above list is in no way limiting, but merely by way of example.

All entities having biological and/or pharmacological activity can be added to this list, provided that the liquid crystals of the invention can improve their actions and/or optimize their stability and/or their solubilities and/or their solutions and/or their bioavailability and/or provide them with better half-lives and/or optimize their duration of actions and/or optimize their efficiencies and/or reduce their adverse effects and/or increase their safety and/or their efficacy and/or their acceptability in living beings.

The antineoplastic agents are drugs intended to block the proliferation of cancer cells—the neoplasm designating a tumor or a cancer.

However, most antineoplastic agents are not drugs with a specific effect on neoplastic cells since they also touch healthy cells. According to a very advantageous aspect of the invention, the present liquid crystals are particularly indicated to optimize the effects and/or acceptability and/or efficacy of the entities of this therapeutic class. The liquid crystals of the invention are used for better solubilizations and/or better stability and/or better safety and/or better half-lives and/or better efficiencies and/or better acceptability and/or to better targeting in living beings.

Antineoplastic agents include several tens of drugs, including alkylating agents, antimetabolites, intercalating agents and antimitotic agents.

The following list is non-exhaustive and each and/or several of its entities may benefit from the advantages of the present liquid crystals.

Antimetabolites: Antifoliques: Methotrexate, Raltrexed, Pemetrexed. Antipyrimidine: Mercaptopurine, Thioguanine, Pentostatin, Cladribine, Fludarabineau antimetabolites: Hydroxycarbamide, Gemcitabine.

Alkylating agents such as nitrogen mustard: Chlorambucil, Melphalan, Chlormethine, Ifosfamide, Cyclophosphamide;

Nitroso-urea: Fotemustine, Lomustine, Carmustine, Streptozocine; Organoplatin: Carboplatin, Cisplatin, Oxaliplatin; Ethylene imine: Thiotepa, Altretamine; Amide Imidazole: Procarbazine, Temozolomide, Dacarbazine; Other alkylating agents: Busulfan, Mitomycin C, Pipobroman.

Intercalating agents: camptothecin, irinotecan, Topotecan anthracycline: Epirubicin, Daunorubicin, Doxorubicin, Pirarubicin, Idarubicin, Mitoxantrone, Amsacrine, Elliptinium, Actinomycin D, Etoposide, Bleomycin

Molecules having an action on the mitotic spindle: Vinca alkaloid, spindle poison, Vinorelbine, Vindesine, Vincristine, Vinblastine;

Taxoids: Paclitaxel, Docetaxel;

Tyrosine kinase inhibitors; VEGFR inhibitors: Sunitinib, Axitinib, Pazopanib; EGFR inhibitors: Afatinib, Erlotinib, Gefitinib, Lapatinib; Inhibitors of BCR-Abl fusion protein: Imatinib, Dasatinib, Nilotinib, Bosutinib, Ponatinib, Sorafenib, Regorafenib; serine-threonine kinase inhibitors RAF: Dabrafenib, Vemurafenib, Sorafenib, Regorafenib; serine-threonine kinase inhibitors MEK: Cobimetinib, TrametinibInhibitors of serine-threonine kinase mTor: Temsirolimus, Eveverolimus; Other tyrosine kinase inhibitors: Crizotinib, Ibrutinib, Iderelalisib, Ruxolitinib

Monoclonal antibodies: Anti-VEGF: Bevacizumab; Anti-EGFR: Cetuximab, Panitumumab; Anti-HER2: Trastuzumab, Pertuzumab; Anti-CD20: Rituximab, Ibritumomab, Ofatumumab; Other monoclonal antibodies: Alemtuzumab, Armatumomab, Daratumumab, Ipilimumab, Panitumumab.

Modified viruses and viruses, oncolytic adenoviruses.

The anti-cancer monoclonal antibodies fall into the category of targeted therapies. They may have a cytolytic effect by fixing a membrane target which allows activation of the complement or recruitment of cytotoxic immune cells. Some have a neutralizing effect, thus preventing the binding of the ligand to its receptor.

The above list is in no way limiting, but merely by way of example.

Accordingly, methods of treatments using one or more agent from the preceding list/paragraph in combination with the liquid crystals of the invention, are claimed.

Such methods of treatments being particularly suitable in related conditions and/or maladies for increasing the safety and/or the efficacy and/or the bioavailability and/or the compliance for a subject in need.

According to the invention, the use of the present crystals in combinations and/or in combinations is particularly useful in the pharmaceutical field, because in addition to their optimized and/or transport activities, they protect sensitive active agents against degradation (enzymatic and/or metabolic) and oxidation.

All entities having biological and/or pharmacological activity can be added to this list, provided that the liquid crystals of the invention can improve their actions and/or optimize their stability and/or their solubilities and/or their bioavailability and/or their best half-lives and/or reduce their adverse effects and/or increase their safety and/or their efficiencies and/or their acceptability in living beings.

In a particularly advantageous aspect of the invention, the present liquid crystals are physiological or substantially physiological due to the fact that the betaine is not a xenobiotic; it will not cause immune reactions and/or allergic reactions and/or metabolic reactions and will thus allow the transport of the cargoes entities without it being neither detected nor metabolizedneither destroyed by the immune system and/or by the enzymatic systems. This will make it possible to increase the therapeutic action of the cargos entities by allowing them to reach their site of specific action in an optimal and/or targeted manner.

In another aspect of the invention, the cargoes entities are encapsulated and/or enveloped and/or housed in a shell consisting of betaine.

In another aspect of the invention, the cargoes entities are, thanks to the present liquid crystals, in the form of molecular dispersions in the solvent.

In an important aspect of the invention, the products obtained are stealth.

In a very advantageous and particular aspect of the invention, the present liquid crystals can be considered as nano-transporters and/or nano-vectors and/or nano-sponges and/or nano-crystals and/or quantum dots and/or fractal structures which due to their physiological characters will have the advantage and the quality of being stealth, that is to say not or hardly detectable by the immune defense systems.

This stealth property represents a notable improvement with respect to PEG (polyethylene glycol) formulations, since the betaine is endogenous and already present naturally in the body, and in view of its non-xenobiotic nature, it will not cause reactions for example of immunity and/or of opsonizations and/or of allergies and/or of rejections because the defense systems recognize it as an endogenous and familiar molecule. Since the cargo entities are housed in these familiar shells/capsules for the immune system, they can reach the target areas and optimize their therapeutic effects.

Likewise, the present liquid crystals and their cargoes can pass the physiological barriers, and in particular the encephalic barrier for optimal deliveries to the brain. In another advantageous aspect of the invention, the betaine having the particularity of being highly bioavailable orally, the present liquid crystals are particularly useful and indicated for transporting entities such as proteins, in particular insulin, in oral formulations. Indeed, the shell and/or the capsule consisting of betaine will allow insulin and/or other entities of interest to escape the enzymatic actions of the gastrointestinal tract while allowing the latter to be usefully absorbed and efficiently directed towards the blood flow.

The oral forms and/or oral formulations of insulins housed/enveloped in the betaine liquid crystals of the invention are claimed.

Likewise, these protective and/or optimizing activities of the present liquid crystals can be applied not only to the cargoes entities listed in the present document, but also to be extended to the RNA and/or DNA sequences and/or to the peptides and/or to the aptamers and/or to the dendrimers and/or to the oligonucleotides for the production of optimal formulationswhether oral and/or parenteral and/or injectable and/or cutaneous and/or pulmonary and/or ophthalmic and/or nasal and/or sublingual and/or administrable via the mucous membranes.

In another advantageous aspect of the invention, the cargo entity must not necessarily have a Log P greater than 1 but can be water-soluble (Log P<1). The liquid crystals of the invention serve for other purposes than solubilization, such as better stability and/or better safety and/or better half-lives and/or better efficiencies and/or better acceptability in living beings. In the general framework of the present invention, when it is a commercial name (that a person skilled in the art readily identifies), the methods of the invention advantageously apply to the crude and/or pure active molecule and/or principles of these medicaments; as well as to their analogues, esters, precursors and metabolites. Nevertheless, if necessary, the methods of the invention can also be applied to their final and/or semi-finished formulations.

According to a particular aspect of the invention, the advantage of using a water-soluble surfactant also lies in the fact that it is possible to produce dry or substantially dry forms after removal and/or evaporation and/or dehydration and/or drying of the solvent (s) (preferably water) of the present liquid crystals. These dry or substantially dry forms will have the advantages of better stability profiles and/or better salting profiles, both in their galenic and/or pharmaceutical forms, than in their absorption and/or action profiles in the living being.

The advantage of the invention is that it can replace the PEG in all known formulations using this molecule, with a notable improvement as to the acceptability by the upper living of the present physiological liquid crystals compared to the PEG.

### Vaccines

According to the invention, the uses of the present crystals are particularly useful in the field of vaccination and vaccines because they provide the antigenic and/or biological materials (DNA, RNA, aptamers) of the vaccines, an envelope and/or a shell which is both protective and which optimizes their outputs and/or their effectiveness at the sites of biological actions.

These activities are useful for live attenuated vaccines, inactivated vaccines, as well as for their (living/inactivated) combinations.

These activities are useful for preventive vaccines, therapeutic vaccines, as well as their combinations (preventives and/or therapeutics).

The present invention which can be carried out at ambient temperature is particularly suitable for the development of vaccines and of products and/or molecules having a sensitivity and/or a lack of stability to heat and/or to caloric excess.

According to a very advantageous aspect of the invention, the present liquid crystals are particularly indicated to optimize the effects and/or the acceptability and/or the effectiveness of the entities of this therapeutic class.

The invention is used for better solubilizations and/or better stability and/or better protections and/or better safety and/or better half-lives and/or better efficiencies and/or better acceptability in living beings.

In an important and original embodiment, when Quillaja Saponaria (E999 or QS-21) is used as the surfactant for forming the present betaine liquid crystals, such liquid crystals will be particularly useful and advantageous to produce new forms of vaccines, due to Quillaja Saponaria established immunostimulant adjuvant properties in vaccines. Accordingly, it establishes a synergistical therapeutical effect between the surfactant and betaine, in addition to the protection offered by present nanocrystals to proteins and nucleotides of interest. Original vaccine formulations can be established based on the preceding features.

### Specific Properties

Moreover, by their mimesis with the lyotropic liquid crystalline phases which are abundant in living systems, in particular the constitutive molecules of the surface of the cell and/or biological membranes, the present crystals can exert a lipid polymorphism and/or exhibit a biomimetic character, and can easily mix with the biological membranes and/or the cell membranes, without high energy requirements during this process.

This will make it possible to deliver “naturally” and/or usefully and/or efficiently the cargo entities of the present invention to the targeted biological sites in an optimal manner.

Likewise, the endocytosis by osmosis and/or passive and/or by active transports can be promoted by the crystals and/or cargoes of the invention, due to betaine established osmotic activities.

In another aspect, the present crystals exert a beneficial activity via the caveolae, for optimal delivery of their cargoes.

The lyotropic liquid crystalline phases are abundant in living systems; it is then referred to as lipid polymorphism. Consequently, the present lyotropic liquid crystals have biomimetic properties reproducing the structure of biological membranes and/or cell membranes, both being liquid crystal forms. Their constituent molecules (for example phospholipids) are perpendicular to the surface of the membrane, but the membrane is flexible. The constituent molecules can be mixed easily, but tend not to leave the membrane because of the high energy requirements of this process.

The present liquid crystals, due to their mimicry with the membranes and/or cells, are capable of better delivery of the cargo entities through and/or to biological and/or cellular membranes. This in addition is facilitated because of the little energy that this process mobilizes by virtue of the present liquid crystals.

### Other Advantages

The present invention constitutes a technological platform making it possible to improve most of the formulations of the prior art. These improvements occur at several levels:
Ease of Industrial Production with Economy of Costs and Means
Well-defined Ingredients having the GRAS status (Generally Recognized as Safe)
Better solubility and therefore better bioavailability
Reduction of the Metabolic Effects of First Passage (EPP)
No food effects due to the absence of lipids
Better targeting of cells and/or tissues
Better safety with fewer non-physiological ingredients
Higher acceptability with the effects of betaine
Stealth Formulations with no opsonizations
Higher therapeutic efficacy, with synergistic effects
Improvements in Metabolism in the Gastrointestinal Tract
Improvements in the Efficiency of Absorptions
Cargoes protections from degradation phenomena leading higher circulating levels.

### Other Aspects of the Invention

According to the invention, the use of the present crystals is particularly useful in the field of cosmetics, because in addition to their optimized activities similar to those brought to the drugs, they allow better skin absorption and transport and protection of sensitive assets that they protect against degradation (enzymatic and/or metabolic) and oxidation.

The present liquid crystals may also be used for therapeutic and/or cosmetic applications such as in Acne, Cellulite, Hair Drop, Sensitive Skin, Creams, Serums, Face and/or Body Masks, Maquillages, Aesthetic Medicine, Thinness, Fragrances, Wrinkles, Hair Health, Anti-Aging Care, Hair Care, Body Care, Face Care, Q10 enzyme, Vitamins, Thinness Care, diets to lose weight.

Chemical uses such as in insecticides, pesticides, antifungal agents for agriculture, petrochemistry. Indeed, many insecticides and pesticides are hydrophobic substances and require uses in high amounts to reach active thresholds, such high doses are toxic to the environment. In a very advantageous aspect, the liquid crystals of the invention allow drastic reductions in the doses of these toxic compounds thus allowing better preservation of the environment.

The present liquid crystals, due to their versatility may also have other industrial applications such as in solar panels, semiconductor and/or superconducting crystals and ferrofluids.

Other electronic uses such as solar panels, quantum computers, LCDs, occulting windows, optical fibers, batteries and energy accumulators (batteries) may be envisaged.

### Water-Soluble Cannabis

The present liquid crystals are particularly useful and effective in solubilizing/protecting lipophilic natural compounds commonly used in drugs and/or in nutraceuticals which comprise phytocannabinoids such as cannabidiol, terpenes, terpenoids, essential oils such as beta-caryophyllene, phyllene, carene, pinene, linalool, limonene, phytol, nerolidol, myrcene, myrcene, fatty acids such as linoleicstearic acid, oleic acid, arachidonoylethanolamide (anandamide), compounds such as co-enzyme Q-10, pterostilbene, lutein, lycopene, other essential oils such as lemon oil, grapefruit seed extract, green tea extract, EGCG, cocoa extract, epigallocatechin gallate, epigallocatechin, epicatechin, catechin, epicatechin gallate, quercetin, curcumin, turmeric, turmeric, d-limonene, lemon oil, carotenoids, astaxanthin or phosphatidylserine.

According to an advantageous aspect of the invention, the present liquid crystals are particularly indicated to optimize the effects and/or acceptability and/or efficacy of the cannabinoids. The liquid crystals of the invention are used for better solubilizations and/or better stability and/or better safety and/or better half-lives and/or better efficiencies and/or better acceptability and/or to better targeting in living beings.

According to a very advantageous aspect of the invention, the present liquid crystals are particularly suitable for solubilizing natural cannabinoids comprising cannabinoids derived from cannabis comprising cannabinoids obtained from a cannabis plant comprising cannabidiol (CBD), cannabidiolic acid (CBDA), cannabinol (CBN), cannabigerol (CBG); cannabigerol monoethylether (CBGM); cannabichromene (CBC); Cannabielsoin (CBE); Cannabicyclol (CBL) Cannabicyclol (CBL); Cannabicitran (CBT); Cannabivarin (CBV); Tetrahydrocannabivarin (THCV); Cannabidivarin (CBDV); Cannabichromethvarin (CBCV); Cannabigerovarin (CBGV); cannabigerol monomethyl ether (CBGM); tetrahydrocannabinol (THC), iso-tetrahydrocannabinol (iso-THC), tetrahydrocannabinol acid (THCA), delta-8-THC, DELTA.9-tetrahydrocannabinol, DELTA.8-tetrahydrocannabinol, 11-hydroxy-tetrahydrocannabinol, 11-hydroxy-DELTA.9-tetrahydrocannabinol, levonantradol. DELTA. 11 tetrahydrocannabinol, tetrahydrocannabivarin, dronabinol, amandamide, nabilone;), the esters thereof, their salts, their metabolites, their mixtures and/or one or more combinations thereof.

The present liquid crystals and/or their crystallized dry forms are used to increase the water solubility of the cannabinoid substances of the preceding paragraph by a factor of 5, preferably by a factor of more than 10, more preferably by more than 50, preferably more than 100, preferably more than 500, more preferably by more than 1000, advantageously in addition to 2000, preferably more than 3000, even more preferentially more than 4000, very preferentially by a factor of more than 5000, said solubility being able to be calculated for example by the USP Paddle method and/or by the dissolved weight of the hydrophobic substance in a given volume of water such as mg/ml (milligram/milliliter).

The invention also describes the use of the present crystals in methods for obtaining and/or extracting (plant leaves) and/or formulations of one or more water-soluble cannabinoids, cannabinoids obtained from a Cannabis plant comprising cannabidiol (CBD), cannabidiolic acid (CBDA), cannabinol (CBN), cannabigerol (CBG); cannabigerol monoethylether (CBGM); cannabichromene (CBC); Cannabielsoin (CBE); Cannabicyclol (CBL)Cannabicyclol (CBL); Cannabicitran (CBT); Cannabivarin (CBV); Tetrahydrocannabivarin (THCV); Cannabidivarin (CBDV); Cannabichromethvarin (CBCV); Cannabigerovarin (CBGV); cannabigerol monomethyl ether (CBGM); tetrahydrocannabinol (THC), iso-tetrahydrocannabinol (iso-THC), tetrahydrocannabinolic acid (THCA), delta-8-THC, DELTA.9-tetrahydrocannabinol. DELTA.8-tetrahydrocannabinol, 11-hydroxy-tetrahydrocannabinol, 11-hydroxy-DELTA.9-tetrahydrocannabinol, levonantradol, DELTA.11-tetrahydrocannabinol, tetrahydrocannabivarin, dronabinol, amandamide, nabilone;), the esters thereof, their salts, their metabolites, their mixtures and/or one or more combinations thereof.

These methods of obtaining and/or extractions described in the preceding paragraph may of course be extended to the other cargo entities described in the present document.

The examples are only to illustrate the invention and do not in any way limit it, since the techniques and/or means of obtaining described herein can be applied to most molecules that pose problems of solubility and/or bioavailability and/or half-life.

### EXAMPLES

The data concerning the ingredients, their Log P and their solubility are extracted all (in a concern of uniformity) of the https://go.drugbank.com website

### Example 1

### Production of Liquid Crystals of Betaine

Procedure: All manipulations and mixtures are carried out at ambient temperatures between 17 & 250 Celsius.

Ingredients: Distilled water (Forever Products), Polysorbate 80 (Origins), anhydrous betaine (Danisco).

Material: Glass Tubes 5 ml, Pipettes VWR 100 μL & 1000 μL, Electronic Balance 0.001 g, Centrifuge 800-1 Centrifugal Machine 4000 rpm (corresponding to 4,000×g), Freezer Candy (−18° C.).

Tube A1: mixing 2 ml of distilled water and 2 g of betaine by vigorous manual stirring, then adding 40 μL of Polysorbate 80 (52 mg) all again manually mixed.

The mixture of B is then subjected for 15 minutes to a centrifugation of 4000 rpm.

Tube B1: mixing 2 ml of distilled water and 40 μL (corresponding to 52 mg) of Polysorbate 80, then adding 2 g of betaine all again mixed by vigorous manual stirring. The mixture 3 is then subjected for 15 minutes to a centrifugation of 4000 rpm.

Tube C1: pouring 2 g of betaine and adding 40 μL of Polysorbate 80 and then 2 ml of distilled water all mixed by vigorous manual stirring. The mixture is then subjected for 15 minutes to a centrifugation of 4000 rpm.

Tube A2: mixing 2 ml of distilled water and 2 g of betaine by vigorous manual stirring, then adding 40 μL of Polysorbate 80 all again manually mixed. Tube not subjected to centrifugation.

Tube B2: mixing 2 ml of distilled water and 40 μL of Polysorbate 80, then adding 2 g of betaine all again mixed by vigorous manual stirring. Tube not subjected to centrifugation.

Tube C2: mixing 2 g of betaine and adding 40 μL of Polysorbate 80 and then 2 ml of distilled water, all being mixed by vigorous manual stirring. Tube not subjected to centrifugation.

### Results

The tubes A2 & C2 are of opaque/milky appearance with a slight foam supernatant, while the tube B2 although being transparent also comprises a foamy supernatant which is greater than for the tubes A2 & C2. The tubes A2, B2 and C2 do not have to the naked eye optical features, except that tube B2 is slightly iridescent.

Conversely, and surprisingly the tubes A1, B1 & C1 are transparent with iridescent and/or opalescent reflections in the bluish and yellow hues, as a function of the direction of the light and/or of the angle of observation.

These remarkable optical characteristics of the A1, B1 & C1 tubes demonstrate birefringence, as well as the liquid crystal nature of the A1, B1 & C1 mixtures.

According to an advantageous aspect of the invention, centrifugation does not allow only the simple mixing of the ingredients which are water-soluble and which should be mixed easily, but it also makes it possible to generate and/or modulate and/or organize and/or direct the orientation of these ingredients by imposing on their mixtures in water a direction and/or a director which arranges them with simple isotropic ingredients (having a single direction) in solution in water in an anisotropic assembly (having several directions).

Under such circumstances, centrifugation gives the mixtures A1, B1 & C1 new characteristics and/or new qualities specific to the liquid crystals.

Here, the director is generated and/or modulated and/or directed by centrifugation.

Embodiments:
Legend: Y = Yes - N = No
Aspects:

| | A2 | B2 | C2 | A1 | B1 | C1 |
|---|---|---|---|---|---|---|
| Milky/Translucid | Y | N | Y | N | N | N |
| Transparent | N | Y | N | Y | Y | Y |
| Iridescent/Nacre | N | N | N | Y | Y | Y |
| Birefringence | N | N | N | Y | Y | Y |

It appears that the use of the same ingredients but in different chronologies and/or conditions of different mixtures leads to diametrically opposite or different results. These differences are even more marked when the concentration of the ingredients is varied, in particular by increasing the concentration of Polysorbate 80 to 100 μL, at 200 μL & at 400 μL, only rare combinations are not milky.

### Example 2

After 3 hours of stability at room temperature (20° C.) of the tubes A2, B2 & C2, without any significant change being involved, both in the (milky or transparent) appearance than that of the supernatants, the 3 tubes are placed for 15 minutes at the freezer at a negative temperature surrounding the minus 180 Celsius.

### Results

Very surprisingly, the contents of the 3 tubes become transparent and acquire the same optical characteristics as those of the tubes A1, B1 & C1 seen previously. The contents A2, B2 & C2 are transparent with iridescent and/or opalescent reflections in the bluish and yellow hues as a function of the direction of the light and/or of the angle of observation.

After cold treatment, remarkably the contents of the tubes A2, B2 & C2 acquire remarkable optical characteristics demonstrating their birefringence, as well as the liquid crystal nature of the mixtures A2, B2 & C2.

### Example 3

Supernatants nevertheless persistent in the tubes A2, B2 & C2 after cold treatment of Example 2, these are immersed in a water bath at 70° Celsius for 5 minutes, which has the effects of completely removing the supernatants.

These results show that it is here the temperature which has organized the orientations of the ingredients in the various mixtures by modulating and/or organizing and/or directing the orientation of these ingredients by imposing on their mixtures in water a direction and/or a director which arranges them of simple isotropic ingredients (having a single direction) in solution in water, into an anisotropic assembly (having several directions). As soon as the temperature treatment gives the mixtures A2, B2 & C2 of the new characters and/or of the new qualities, specific to the liquid crystals.

Even more remarkably, the liquid crystals of the invention possess and demonstrate characters and/or qualities of thermotropic liquid crystals.

### Example 4

### Germinations/Nucleations on Drying.

Tube A: mixing 2 ml of distilled water and 2 g of betaine mixed by vigorous manual stirring. The mixture of the 2 is then subjected for 15 minutes to a centrifugation of 4000 rpm.

Tube B: mixing 2 ml of distilled water and 40 μL of Polysorbate 80 (55 mg) and then mixing by vigorous manual stirring, then adding 2 g of betaine, all of which is mixed manually. The mixture of B is then subjected for 15 minutes to a centrifugation of 4000 rpm.

Results: The tube B is transparent nacred confirms a bluish hue of birefringence.

The tube A remains transparent without any apparent hue.

The 2 tubes are poured into glass Petri dishes (35 mm diameter) and subjected to drying at 60° C. in a hot air dehydrator (Stockli).

The content of the tube A is dried into crystals which remain confined in the initial volume of the solution. The germination/nucleation being of the homogeneous primary type for the solution of betaine alone.

The content of Tube B undergoes germination/nucleation of the heterogeneous primary type with germs forming on the walls of the Petri dish and then overflowing the dish as can be seen in FIG. 1.

This nucleation rates of the content of the tube B, much greater than this of tube A, was completely unexpected. The addition to betaine solution of surfactant having HLB>12, in the proportions of the invention, leaded to an unexpected, spectacular and obvious result which a single glance to FIG. 1 can certify.

Comparatively to the simple betaine solution of Tube A, it is clear that the new betaine liquid crystals of the invention, thanks to their optical properties (bluish and iridescent—birefringent), their particular germination/nucleation profile, as to their unique solubilizing potency, are not mentioned nor evoked by the previous published art.

Iridescence and birefringence are the established hallmarks of liquid crystals nanoformulations, bluish iridescence the one for quantum dot formulations.

### Example 5

Solubilization of Curcumin—Log P 4.12—Solubility in Water 0.00575 mg/ml

Mixing 2 ml of distilled water and 40 μL of Polysorbate 80, then adding 400 mg of pure curcumin, manually mixing and then adding 2 g of betaine all again mixed by vigorous manual stirring. The mixture of the 4 ingredients is then subjected for 20 minutes to a centrifugation of 4000 rpm.

A pellet representing 30% to 40% of the insolubilized curcumin remains in the bottom of the tube. The remaining fraction is completely solubilized and gives a transparent solution of deep amber brown color without any suspended particle. 400 μl of the solution produced are taken and poured into a tube of 5 ml of distilled water (pH 7). The solution immediately solubilizes and gives a transparent solution with a bright yellow color. The solution stored in the absence of light shows no changes over time (24 h).

400 μl of the solution produced are taken and poured into a tube of 5 ml of acetic acid (pH 2.2). The solution immediately solubilizes and gives a transparent solution with a bright yellow color. The solution stored in the absence of light shows no changes over time (24 h).

The mother solution obtained after centrifugation is estimated at a concentration of 80 mg/ml of curcumin (weighted at 60 mg/ml-base). It remains stable without any deposition for long periods of time.

The solubility of curcumin in water (20° C.) was multiplied by a factor of 10,000.

### Example 6

Solubilization of Fenofibrate—Log P 5.28—Solubility in Water 0.000707 mg/ml

Mixing 2 ml of distilled water and 40 μL of Polysorbate 80, then adding 1 ml of ethanol and then 150 mg of Fenofibrate, mixing manually then adding 2 g of betaine all again mixed by vigorous manual stirring. The mixture of the 4 ingredients is then subjected for 30 minutes to a centrifugation of 4000 rpm.

A base representing 20% of the non-solubilized fenofibrate remains in the bottom of the tube. The remaining fraction is completely solubilized and gives a transparent solution of yellow/green color without any suspended particle.

The mother solution obtained after centrifugation is estimated at a concentration of 27 mg/ml of fenofibrate (weighted at 20 mg/ml-base). It remains stable without any deposition for long periods of time. Poured into a water glass it dissolves immediately.

The water solubility of fenofibrate (20° C.) was multiplied by a factor of 28.000.

### Example 7

Solubilization of Fenofibrate—Log P 5.28—Solubility in Water 0.000707 mg/ml

Mixing 2 ml of distilled water and 40 μL of Polysorbate 80, then adding 100 mg of Fenofibrate, mixing manually then adding 2 g of betaine all again mixed by vigorous manual stirring. The mixture of the 4 ingredients is then subjected for 40 minutes to a centrifugation of 4000 rpm.

A base representing 35% of the non-solubilized fenofibrate remains in the bottom of the tube. The remaining fraction is completely solubilized and gives a transparent solution of green/yellow color without any suspended particle.

400 μl of the solution produced are taken and poured into a tube of 5 ml of distilled water (pH 7). The solution immediately solubilizes and gives a transparent solution with yellow/green reflections. The solution stored in the absence of light shows no changes over time (24 h).

400 μl of the solution produced are taken and poured into a tube of 5 ml of acetic acid (pH 2.2). The solution immediately solubilizes and gives a transparent solution with yellow/green reflections. The solution stored in the absence of light shows no changes over time (24 h).

The mother solution obtained after centrifugation is estimated at a concentration of 20 mg/ml of fenofibrate (weighted at 15 mg/ml-base). It remains stable without any deposition for long periods of time.

The solubility of fenofibrate in water was multiplied by a factor of 20.000.

### Example 8

Solubilization of Cannabidiol—Log P 6.1—Solubility in Water 0.0126 mg/ml Mixing 2 ml of distilled water and 40 μL of Polysorbate 80, then adding 160 mg of cannabidiol (CBD), mixing manually then adding 2 g of betaine all again mixed by vigorous manual stirring. The mixture of the 4 ingredients is then subjected for 30 minutes to a centrifugation of 4000 rpm.

Cannabidiol is completely solubilized and gives a transparent solution of golden color with bluish reflections, without any suspended particle.

200 μl of the solution produced are taken and poured into a tube of 5 ml of distilled water (pH 7). The solution immediately solubilizes and gives a transparent solution with mauve/dew reflections. The solution stored in the absence of light shows no changes over time (24 h).

200 μl of the solution produced are taken and poured into a tube of 5 ml of acetic acid (pH 2.2). The solution immediately solubilizes and gives a transparent solution with mauve/dew reflections. The solution stored in the absence of light shows no changes over time (24 h).

The mother solution obtained after centrifugation is estimated at a concentration of 47 mg/ml of Cannabidiol (not weighted because no pellet). Sheltering light, it is remained stable without any deposition, or modification for a year.

Knowing that the solubility in water of Cannabidiol is 0.0126 mg/ml, the 47 mg/ml obtained correspond to an increase in its solubility by a factor of 3730 times.

### Example 9

Drying and Germinating of the Combinations.

The liquid crystals as in Examples 5, 6 & 7 are produced.

The three tubes are poured into glass Petri dishes (diameter 35 mm) and subjected to drying at 50° C. in hot air dehydrator (Stockli).

Results

The contents of the 3 tubes undergo nucleation/nucleation of the heterogeneous primary type with germs forming on the walls of the Petri dishes and then overflowing the boxes, in a manner similar to FIG. 1.

The curcumin liquid crystal adopts a bright scarlet/yellow color.

The fenofibrate liquid crystal adopts a blanket/greenish color.

The cannabidiol liquid crystal adopts a pink color to the mauve, the hues of which are similar to the cannabis flower.

### Example 10

The 3 dry products of Example 9 are ground until reaching±50 Mesh and stored in the absence of light and moisture for 6 months.

### Example 11

Visual Tests of Fenofibrate, Curcumin & CBD solutions.

After the 6 months, the 3 dry powders of example 9 are solubilized at a rate of a gram of dry powder each in 20 centiliters of pure water (pH 7).

The 3 dry powders redissolve immediately (a few seconds) by giving solutions without particles in suspension, and without deposits or supernatants.

The pH of the same 3 solutions is then varied from the neutral to the starting pH (dosage form, tablet, capsule, or the like) at acidic pH by adding acetic acid up to pH 2.5 (gastric environment) and then these solutions are neutralized by adding sodium bicarbonate to pH 8 (intestinal environment).

The 3 solutions show no change in their aspects, nor in their shades. There is no precipitation.

### Example 12

100 mg of dry powder of Curcumin of the preceding example are dissolved in 100 ml of pure water before being kept in the dark for 14 days. This solution is then evaluated with DLS (Dynamic Light Scattering) in a Zetasizer apparatus (University of Namur).

The average particle size of the solution is within 10 nanometers, or even less, with a very low polydispersity.

### Example 13

Solubilization of Clofazimine Log P—7.39—Solubility in Water 0.00151 mg/ml

Mixing 2 ml of distilled water and 35 μL of Polysorbate 80, then adding 50 mg of Clofazimine, mixing manually and then subjecting to a centrifugation of 4000 rpm and then adding 2 g of betaine all again mixed by vigorous manual stirring. The mixture of the 4 ingredients is then subjected for 30 minutes to a centrifugation of 4000 rpm.

Clofazimine is completely solubilized and gives a transparent solution of yellow (honey) yellow color, without any suspended particle.

250 μl of the solution produced are taken and poured into a tube of 5 ml of distilled water (pH 7). The solution immediately solubilizes and gives a transparent solution with slight red reflections. The solution stored in the absence of light shows no changes over time (24 h).

250 μl of the solution produced are taken and poured into a tube of 5 ml of acetic acid (pH 2.2). The solution immediately solubilizes and gives a transparent solution with a red color carmine. The solution stored in the absence of light shows no changes over time (24 h).

The mother solution obtained after centrifugation is estimated at a concentration of 14.7 mg/ml of Clofazimine (not weighted because no pellet).

Knowing that the solubility in water of Clofazimine is 0.00151 mg/ml, 14.7 mg/ml obtained correspond to an increase in its solubility by a factor of 9730 times.

### Example 14

The remaining solution of the preceding example is poured into a glass Petri dish (diameter 35 mm) and subjected to drying at 60° C. in a hot air dehydrator (Stockli).

Results

Upon drying, the solution undergoes germination/nucleation of heterogeneous primary type with germs forming on the walls of the Petri dish and then overflowing from the box in a similar manner to Tube B in FIG. 1.

The color of the crystal formed is red orange.

The dry product is then ground until it reaches±50 Mesh and stored in the absence of light and moisture for 10 days.

Next, 100 mg of dry powder are taken and poured into a tube of 5 ml of distilled water (pH 7). The solution immediately solubilizes and gives a red/pink transparent solution. The solution stored in the absence of light shows no changes over time (24 h).

Next, 100 mg of dry powder are taken and poured into a tube of 5 ml of acetic acid (pH 2.2). The solution immediately solubilizes and gives a red carmine transparent solution. The solution stored in the absence of light shows no changes over time (24 h).

Then this acidic solution is brought to an alkaline pH of 8 by adding sodium bicarbonate, in order to mimic the conditions of the pH differences encountered in the stomach and then the intestine.

The solution remains stable without any precipitation or modification, showing by the stability of the products of the invention and their utility in particular for oral uses, as well as any other routes of administrations known in the art.

What I claim is:

1. A glycine betaine based liquid crystal comprising in dry matter at least:
80 to 99.5% by weight of glycine betaine of formula (CH₃)₃N⁺CH₂COO⁻, CAS number: 107-43-7, and
0.5 to 20% by weight of a non-ionic water soluble tensioactive system selected from the group consisting of non-ionic water soluble tensioactive agents having a HLB value equal to 12 or more than 12 and mixtures of non-ionic water soluble tensioactive agents having each a HLB value equal to 12 or more than 12,
whereby the weight ratio glycine betaine/non-ionic water soluble tensioactive system is greater than 10:1,
said glycine betaine and said non-ionic water soluble tensioactive being solubilized in water with a weight ratio glycine betaine/water comprised between 1:3 to 3:1 for forming the glycine betaine based liquid crystal.

2. The glycine betaine based liquid crystal of claim 1 having a particle size of less than 350 nanometers.

3. The glycine betaine based liquid crystal of claim 1 having a particle size of less than 100 nanometers.

4. The glycine betaine based liquid crystal of claim 1 having a particle size of less than 20 nanometers.

5. The glycine betaine based liquid crystal of claim 1, in which the weight ratio glycine betaine/non-ionic water soluble tensioactive system is comprised between 20:1 and 50:1.

6. The glycine betaine based liquid crystal of claim 1, in which the non-ionic water soluble tensioactive system is selected from the group consisting of non-ionic water soluble tensioactive agents having a HLB value equal to or more than 13 and mixtures of non-ionic water soluble tensioactive agents having each a HLB value equal to or more than 13.

7. The glycine betaine based liquid crystal of claim 1, in which the non-ionic water soluble tensioactive system is selected from the group consisting of Tween 20 (E432-HLB 15), Tween 80 (E433-HLB 15), Tween 40 (E434-HLB 15.6), Tween 60 (E435-HLB 14.9), Tween 65 (E436-HLB 10.5), Quillaja Saponaria Molina (E999), as well as their mixtures.

8. The glycine betaine based liquid crystal of claim 1 submitted to drying step for reducing the water content to less than 10% by weight.

9. The glycine betaine based liquid crystal of claim 1, which further comprises a therapeutic entity, whereby the weight ratio glycine betaine/therapeutic entity is equal to or greater than 10:1.

10. The glycine betaine based liquid crystal of claim 9, in which the therapeutic entity has a octanol/water partition coefficient defined by a Log Kow greater than 1.5.

11. The glycine betaine based liquid crystal of claim 9, in which the therapeutic entity is selected from the group consisting of dietetic substances, cosmetic substances, nutrients and pharmaceutical substances.

12. The glycine betaine based liquid crystal of claim 1, which is substantially free of organic solvent.

13. A composition for administering to a mammal a therapeutic entity, said composition comprising:
glycine betaine based liquid crystals comprising in dry matter at least:
80 to 99.5% by weight of glycine betaine of formula (CH₃)₃N⁺CH₂COO⁻, CAS number: 107-43-7,
0.5 to 20% by weight of a non-ionic water soluble tensioactive system selected from the group consisting of non-ionic water soluble tensioactive agents having a HLB value equal to 12 or more than 12 and mixtures of non-ionic water soluble tensioactive agents having each a HLB value equal to 12 or more than 12, and
a quantity of the hydrophobic entity,
whereby the weight ratio glycine betaine/non-ionic water soluble tensioactive system is greater than 10:1, and whereby the weight ratio glycine betaine/hydrophobic entity is equal to or greater than 5:1,
said glycine betaine and said non-ionic water soluble tensioactive being solubilized in water with a weight ratio glycine betaine/water comprised between 1:3 to 3:1 for forming the glycine betaine based liquid crystal.

14. The composition of 13, in which the glycine betaine based liquid crystals have a particle size of less than 350 nanometers.

15. The composition of 13, in which the glycine betaine based liquid crystals have a particle size of less than 100 nanometers.

16. The composition of 13, in which the glycine betaine based liquid crystals have a particle size of less than 20 nanometers.

17. The composition of 13, in which the weight ratio glycine betaine/non-ionic water soluble tensioactive system of the glycine betaine based liquid crystals is comprised between 20:1 and 50:1.

18. The composition of 13, in which the non-ionic water soluble tensioactive system of the glycine betaine based liquid crystals is selected from the group consisting of non-ionic water soluble tensioactive agents having a HLB value equal to or more than 14 and mixtures of non-ionic water soluble tensioactive agents having each a HLB value equal to 13 or more than 13.

19. The composition of 13, in which the non-ionic water soluble tensioactive system of the glycine betaine based liquid crystals is selected from the group consisting of Tween 20 (E432-HLB 15), Tween 80 (E433-HLB 15), Tween 40 (E434-HLB 15.6), Tween 60 (E435-HLB 14.9), Tween 65 (E436-HLB 10.5), Quillaja Saponaria Molina (E999), as well as their mixtures.

20. The composition of claim 13 having a water content of less than 10% by weight.

21. The composition of claim 13 having a water content of less than 1% by weight.

22. The composition of claim 13, in which the therapeutic entity has an octanol/water partition coefficient defined by a Log Kow greater than 1.5.

23. The composition of claim 22, in which the therapeutic entity has an octanol/water partition coefficient defined by a Log Kow greater than 3.

24. The composition of claim 13, which is substantially free of organic solvent.

25. The composition of claim 13, in which the glycine betaine based liquid crystals are in a powdery form.

26. The composition of claim 13, being in a form selected from the group consisting of
(a) ready to use administration forms selected from the group consisting of oral formulations, sublingual formulations, rectal formulations, parenteral formulations, topic formulations, injectable formulations, mucosal formulations, nasal formulations, cutaneous formulations, subcutaneous formulations, transcutaneous formulations, transdermal formulations, pulmonary formulations, eye drop formulations, and ready to use administration forms for preparing formulation, and
(b) intermediate administration forms adapted to be mixed with water for preparing at least one ready to use administration forms selected from the group consisting of oral formulations, sublingual formulations, rectal formulations, parenteral formulations, topic formulations, injectable formulations, mucosal formulations, nasal formulations, cutaneous formulations, subcutaneous formulations, transcutaneous formulations, transdermal formulations, pulmonary formulations, eye drop formulations.

27. A process for the preparation of a glycine betaine based liquid crystal comprising in dry matter at least:
80 to 99.5% by weight of glycine betaine of formula (CH₃)₃N⁺CH₂COO⁻, CAS number: 107-43-7, and
0.5 to 20% by weight of a non-ionic water soluble tensioactive system selected from the group consisting of non-ionic water soluble tensioactive agents having a HLB value equal to or more than 12 and mixtures of non-ionic water soluble tensioactive agents having each a HLB value equal to 12 or more than 12,
whereby the weight ratio glycine betaine/non-ionic water soluble tensioactive system is greater than 10:1.
said process comprising at least the following steps:
a preparation step of an aqueous medium comprising water, the glycine betaine and the tensioactive system, whereby in dry matter the aqueous medium comprises at least:
80 to 99.5% by weight of glycine betaine of formula (CH₃)₃N⁺CH₂COO⁻, CAS number: 107-43-7, and
0.5 to 20% by weight of a non-ionic water soluble tensioactive system selected from the group consisting of non-ionic water soluble tensioactive agents having a HLB value equal to 12 or more than 12 and mixtures of non-ionic water soluble tensioactive agents having each a HLB value equal to 12 or more than 12,
whereby the weight ratio glycine betaine/non-ionic water soluble tensioactive system is greater than 10:1,
said glycine betaine and said non-ionic water soluble tensioactive being solubilized in water with a weight ratio glycine betaine/water comprised between 1:3 to 3:1, and
a mixing step of the aqueous medium for forming the glycine betaine based liquid crystal.

28. The process of claim 27, in which the mixing step is operated for generating glycine betaine based liquid crystals with a particle size of less than 350 nanometers,

29. The process of claim 27, in which the mixing step is operated for generating glycine betaine based liquid crystals with a particle size of less than 100 nanometers,

30. The process of claim 27, in which the weight ratio glycine betaine/non-ionic water soluble tensioactive system in the aqueous medium is comprised between 20:1 and 50:1.

31. The process of claim 27, in which the non-ionic water soluble tensioactive system of the aqueous medium is selected from the group consisting of non-ionic water soluble tensioactive agents having a HLB value equal to 13 or more than of more than 13 and mixtures of non-ionic water soluble tensioactive agents having each a HLB value equal to or more than 13.

32. The process of claim 27, in which the aqueous medium has a betaine content such that the weight ratio betaine/water is about 1:1.

33. The process of claim 27, in which the preparation step of the aqueous medium comprises a step of adding a therapeutic entity, whereby the weight ratio betaine/therapeutic entity is greater than 10:1.

34. The process of claim 33, in which the aqueous medium consists of water, the glycine betaine, the non-ionic water soluble tensioactive system and the therapeutic entity, whereby the therapeutic entity has a octanol/water partition coefficient defined by a Log Kow greater than 1.5,
whereby the therapeutic entity of the glycine betaine based liquid crystals of the composition has a solubility in water which is at least 100 times higher than the solubility of the hydrophobic therapeutic entity as such in water.

35. The process of claim 33, in which the therapeutic entity is selected from the group consisting of dietetic substances, cosmetic substances, nutrients and pharmaceutical substances.

36. The process of claim 27, in which the preparation step of the aqueous medium comprises a step of adding a hydrophobic entity, whereby the weight ratio betaine/therapeutic entity is greater than 10:1,
in which the aqueous medium consists of water, the glycine betaine, the non-ionic water soluble tensioactive system and the hydrophobic entity, whereby the hydrophobic entity has a octanol/water partition coefficient defined by a Log Kow greater than 1.5,
in which the therapeutic entity is selected from the group consisting of dietetic substances, cosmetic substances, nutrients and pharmaceutical substances,
whereby the preparation step of the aqueous medium comprises the following steps:
mixing the water, the non-ionic water soluble tensioactive system and the hydrophobic entity, so as to prepare an intermediate aqueous medium, and
adding the glycine betaine in dry form to the intermediate aqueous medium.

37. The process of claim 27, which further comprises at least one process step selected from the group consisting of:
a centrifugation step,
a shear mixing step,
a drying step for removing at least partly water of the aqueous medium,
a separation step for removing at least partly the hydrophobic entity not in a solubilized form present in the aqueous medium,
a grinding step, and
a shaping step with a carrier.

38. A method of treatment of a mammal selected from the group consisting of:
(a) mammals suffering at least one trouble from a disease, and
(b) mammals at risk of suffering at least one trouble from a disease, by administering to said mammal at least a dose of a therapeutic entity selected from the group consisting of nutrients, pharmaceutical substances and mixtures thereof,
in which the therapeutic entity to be administered is in the form of a composition comprising
glycine betaine based liquid crystals comprising each in dry matter at least:
80 to 99.5% by weight of glycine betaine of formula (CH₃)₃N⁺CH₂COO⁻, CAS number: 107-43-7,
0.5 to 20% by weight of a non-ionic water soluble tensioactive system selected from the group consisting of non-ionic water soluble tensioactive agents having a HLB value equal to 12 or more than 12 and mixtures of non-ionic water soluble tensioactive agents having each a HLB value equal to 12 or more than 12, and
a quantity of the hydrophobic entity,
whereby the weight ratio glycine betaine/non-ionic water soluble tensioactive system is greater than 10:1, and whereby the weight ratio glycine betaine/therapeutic entity is equal to or greater than 5:1,

39. The method of treatment of claim 38, in which the composition for administering the therapeutic entity in a form selected from the group consisting of oral formulations, sublingual formulations, rectal formulations, parenteral formulations, topic formulations, injectable formulations, mucosal formulations, nasal formulations, cutaneous formulations, subcutaneous formulations, transcutaneous formulations, transdermal formulations, pulmonary formulations, and eye drop formulations.
